(19) United States
(12) Patent Application Publication
(10) Pub. No.: US 2002/0142347 A1
(43) Pub. Date: Oct. 3, 2002

(54) Biosensor

(75) Inventors: Jens Knudsen, Aarslev (DK); Maiken Camilla Trolle Wadum, Odense SO (DK); Jens Villadsen, Odense S (DK); Thomas B.F. Neergaard, Odense SO (DK)

Correspondence Address: BROWDY AND NEIMARK, P.L.L.C., 624 NINTH STREET, NW, SUITE 300, WASHINGTON, DC, 20001-5303, US

(73) Assignee: BIOSENSOR ApS, Aarslev (DK)

(21) Appl. No.: 09/987,108

(22) Filed: Nov. 13, 2001

Related U.S. Application Data
(60) Provisional application No. 60/262,366, filed on Jan. 19, 2001.

(30) Foreign Application Priority Data
Nov. 10, 2000 (DK) PA 2000 01683
Nov. 10, 2000 (DK) PA 2000 01638

(51) Int. Cl. G01N 33/543; G01N 33/537; G01N 33/53; A61K 38/00; A01N 37/18; C07K 2/00; C07K 4/00; C07K 5/00; C07K 7/00; C07K 14/00; C07K 16/00; C07K 17/00

(52) U.S. Cl. 435/7.1; 514/2; 530/300

(57) ABSTRACT

The invention relates to a biochemical assay for wide class of hydrophobic Coenzyme A esters wherein the analyte is caused to react with a specifically binding, modified protein, and thereby causing a detectable signal. A one step assay for hydrophobic carboxylic acid esters in whole blood, serum, food and feed preparations, tissue extracts, acyl-CoA synthetase reaction media and various laboratory conditions using a modified Coenzyme A- and acyl-CoA binding protein (ACBP) is provided. Furthermore the invention relates to a construct comprising a peptide and a signal moiety for performing an assay, a kit for assaying hydrophobic CoA esters, hydrophobic carboxylic acids, triacylglycerides, phospholipids, and cholesterolesters.

The invention relates to a biochemical assay for wide class of hydrophobic Coenzyme A esters wherein the analyte is caused to react with a specifically binding, modified protein, and thereby causing a detectable signal. A one step assay for hydrophobic carboxylic acid esters in whole blood, serum, food and feed preparations, tissue extracts, acyl-CoA synthetase reaction media and various laboratory conditions using a modified Coenzyme A- and acyl-CoA binding protein (ACBP) is provided. Furthermore the invention relates to a construct comprising a peptide and a signal moiety for performing an assay, a kit for assaying hydrophobic CoA esters, hydrophobic carboxylic acids, triacylglycerides, phospholipids, and cholesterolesters and a nucleotide sequence encoding the peptide as well as an expression vector and a cell comprising the nucleotide sequence.

### BACKGROUND OF THE INVENTION

An obligatory step in beta-oxidation, incorporation in to complex lipids or modification of fatty acids in living cells is conversion to its Coenzyme A thioester derivative (acyl-CoA). Besides playing a key role in lipid metabolism acyl-CoA esters have also been shown to act as regulatory molecules regulating enzyme activities, vesicular transport, hormone signalling, Ca²⁺ flux, ionchannels and the rate of transcription of specific genes (Færgeman & Knudsen, 1997; Biocem. J 323, p 1-12).

Long chain free fatty acid (FFA) with acyl chains >16 carbons are quantitatively the most important physiological energy source. The concentration of FFA in growth media and circulating blood is the rate determining factor in regulation of fatty acid uptake (Glatz and van der Vusse, 1996; 35, 243-282) and have been shown to affect intracellular acyl-CoA concentrations (Sterchele, et. al.1994; Biochem. Pharm.48, 955-966). Although fatty acids and acyl-CoA esters are important and essential for normal physiological function they are also potent modulators of cellular activity (Færgeman & Knudsen,1997; Biocem.J 323, 1-12). Dietary fatty acids, through their influence on circulating fatty acid and intracellular acyl-CoA levels and composition, specifically modulate the onset of various diseases including cancer (Cave W. T., 1991; FEBS2166; Welsch C. W. 1992; Cancer Res. Suppl. 52, 2040-2048), atherogenesis (Chin, J. P., 1994; Prost. Leuk, Essent. Fatty Acids, 50, 211-222), hyperlipidemia (Grundy and Denke, 1990; J. Lipid. Res. 31, 1149-1172), insulin resistance (Storlien, L. H., 1987; Science 237, 885-888) and hypertension (Moris, et. al., 1993; Circulation 88, 523-533).

In many instances determination of total fatty acids levels is of significant importance in diagnosis and treatment of disease or studying the mechanisms causing it. For example fatty acids are believed to be an important factor in the cause of ventricular arrhythmiaes during acute myocardial infarction (Makiguchi M, et. al. (Japan) Jul 1988, 63, 624-634). Differences in circulating levels of fatty acid are found in AIDS patients (Christeff, et. al., 1988 Eur. J. Cancer. Clin. Oncol. 24, 1179-1183). Plasma fatty acid concentrations in non insulin dependent diabetes mellitus are believed to be indicative for insulin resistance (Fraze et. al. 1985; J. Clin. Endocrinol. Metab. 61, 807-811). Fatty acid have been implicated in pathogenesis of thromboatherosclerosis, (Travella et al, 1985, Nutr. Res. 5, 355-65). Elevated levels of fatty acids have been found in human cancer patients and animal models (Storlien, L. H., 1987; Science 237, 885-888).

Because the circulating level of FFA influences the intracellular level of acyl-CoA esters, these esters could play an essential role in mediating regulatory and pathogenic effects of increased circulating FFA in various diseases mentioned above.

These effects could involve regulation of acetyl-CoA carboxylase, AMP-activated kinase-kinase, mitochondrial acyl-CoA synthetase, citrate transporter, HMG-CoA reductase, carnitin palmitoyl-CoA transferase, long-chain acyl-CoA dehydrogenase, hormone sensitive lipase, adenine nucleotide translocase, glucokinase, glucose-6-phosphate dehydrogenase, glucocose-6-phosphatase, pyruvate dehydrogenase, Ca²⁺ release from and uptake in intracellular stores, sodium/potassium ATPase, ATP sensitive potassium channels, protein kinase C, nuclear thyroid hormone receptor, vesicular transport, and proteolysis (see Færgeman and Knudsen, Biochem J. 323, 1-12 1997 for review)

Long chain acyl-CoA esters are highly amphiphatic molecules, which bind unspecifically to proteins, test tube walls and they partition into lipid membranes (k=1.5×10⁵, Færgeman and Knudsen, 1997, Biochem J., 323, 1-12). The concentration of free unbound acyl-CoA esters which is the regulatory species is therefore very different from the total concentration. Measurement of free long chain acyl-CoA in vivo and in vitro therefore has important applications in a wide variety of biochemical, biophysical, cell biologic and physiological research. Various HPLC, GC and enzymatic methods for determination of total acyl-CoA levels in tissue extracts and body fluids have been developed. However, no method is yet available for determining free acyl-CoA levels in tissue extracts, body fluids or in cytosol of living cells (Bækdal et. al. 1996; Advances in Lipid Methodology - Three, 109-131, Editor, Christie W. W., The Olie Press, Dundee Scotland, UK, for review).

A number of methods for determination of total fatty acid in blood, body fluids, cell culture media have been developed. One set of methods require extraction by organic solvent essentially as described by Folch et. al., 1957 (J. Biol. Chem. 226, p 497). The extracted fatty acids are subsequently quantified by gas-chromatography after methylation (Baty, and Pazouki, 1987 Chromatography, 395, p 403), by complexing to 63Ni in the organic phase in a two phase system (Iio, R. J., 1970, Anal. Biochem. 36, p105) or by HPLC after derivatisation with a UV absorbing group (Miwa et. al., 1987, J. Chromatography 416, p 237). In another method (U.S. Pat. No. 4,491,631) fatty acids are converted to acyl-CoA esters by acyl-CoA synthetase and quantified in an enzyme linked acyl-CoA dehydrogenase assay. This assay has recently been marketed in a different version where the acyl-CoA formed by the acyl-CoA synthetase is oxidised by acyl-CoA oxidase and the formed H₂O₂ is quantified by reaction with 3-methyl-N-ethyl-N-(beta-hydroxyethyl)-aniline to give a dye (Wako Chemicals USA, Inc.Richmont, Va. 23237, USA).

Very recently, a method for determination of free fatty acids in blood using fluorescently modified fatty acid binding protein (ADIFAB) has been developed (Richieri et al, J. Biol. Chem.267, 23495-23501, 1992, U.S. Pat. No. 5,470,714). This method can also be used to calculate total circulating total fatty acid concentration if the serum albumin concentration and its binding properties are know (Richieri et al, Molecular and Cellular Biochemistry, 192: 87-94, 1999). A disadvantage of this assay is that the binding between the analyte and the sensor is not very strong. The dissociation constant, K_{D}, for the complex between ADIFAB and various common fatty acids (palmitate, oleate, linileate, arachidonate, linolenate) lies in the range of 0.28 to 2.5 μM. In the presence of albumin (which is present in all blood samples) in the sample, the fatty acids bind to both ADIFAB and albumin. Thus, in order to make a reliable estimate of the concentration of free fatty acid, the concentration of albumin in the sample must also be known. Furthermore, fatty acids have a high affinity to surfaces, especially to plastic surfaces. In an assay based on fatty acid binding protein, both the protein, albumin and any plastic surfaces will compete for the free fatty acids with approximately the same affinity resulting in rather unpredictable results.

In U.S. Pat. No. 5,512,429 (BRITISH TECHNOLOGY GROUP LTD.) another method for selectively measuring fatty acids using a probe is disclosed. The disclosure more specifically concerns a method for assaying an enzyme being capable of releasing fatty acids from a substrate or for assaying fatty acids. According to the method described, serum albumin is first removed from the sample (which is most often a serum sample). The enzyme activity or the concentration of the fatty acid is measured by measuring the binding of the fatty acids to a fatty acid binding protein. According to the disclosure the binding should be a binding with a dissociation constant of 10⁻⁵M or less. The method for detecting the fatty acid-FABP binding is by a competition assay with a known amount of a radioactively labelled fatty acid. In a particularly preferred embodiment, the label is a polycyclic fluorophore, especially a naphtalene or anthracene having a polarity-sensitive fluorescent group. As the label moves from a polar to a non-polar environment, the fluorescent group undergoes a change in fluorescent emission.

In a later publication by the same authors (U.S. Pat. No. 5,449,607 (BRITISH TECHNOLOGY GROUP INC.)) it is asserted that there is no need for removal of albumin prior to performing the assay due to the high specificity of the binding. This may be possible by standardisation of the amount of albumin in the samples and the calibration samples. Under all circumstances it is inevitable that albumin competes with FABP for the free fatty acids and that albumin thus binds at least a fraction of the free fatty acids in the sample.

In general, the prior art methods for measuring the concentration of fatty acids and related compounds through binding assays are characterised by low precision due to the relatively low affinity for fatty acids and low selectivity, since fatty acid binding proteins have a considerable affinity towards almost any hydrophobic compound of a certain size.

### SUMMARY OF THE INVENTION

A first aspect of the invention relates to a method for determination of the concentration of free unbound hydrophobic Coenzyme A ester in a sample comprising the steps of

providing a hydrophobic Coenzyme A binding construct exhibiting a first signal when unbound and exhibiting a measurably different second signal when bound to a hydrophobic Coenzyme A ester,

contacting the sample with the labelled hydrophobic Coenzyme A binding construct,

allowing at least one species of unbound free hydrophobic Coenzyme A ester to bind to the hydrophobic Coenzyme A binding construct forming a complex comprising a hydrophobic Coenzyme A ester and the hydrophobic Coenzyme A binding construct,

detecting a signal from the complex,

correlating the signal to the concentration of the at least one species of hydrophobic Coenzyme A ester in the sample.

The method according to the present invention provides an easy, rapid and yet highly specific and accurate method for measuring the concentration of hydrophobic Coenzyme A esters.

All compounds that can be converted to hydrophobic CoA esters can be measured indirectly by the method according to the invention by combination with suitable reactions for hydrolysis and Coenzyme A thioesterification. Such compounds include but are not limited to free fatty acids, lipids, triacylglycerides, phospholipids, cholestrolesters.

A great advantage of the method according to the present invention is the high affinity of the peptide comprised in the construct for hydrophobic CoA esters. The K_{D} of the construct with respect to hydrophobic CoA esters is preferably several orders of magnitude lower than the affinity of prior art constructs used for binding of fatty acids. Due to the increased binding affinity the interference of other potential sinks for hydrophobic CoA esters such as albumin, Fatty Acid Binding Proteins or plastic surfaces with the binding assay is markedly reduced. The result is a much more precise estimation of the concentration of the hydrophobic CoA esters than hitherto possible.

Another advantage of the method according to the present invention is that the peptide part of the construct is extremely selective in its binding and binds in reality only CoA and hydrophobic CoA esters. The binding affinity of the constructs for other hydrophobic compounds such as free fatty acids, is extremely low and the presence of other hydrophobic compounds in the sample thus does not interfere with the assay according to the invention. Thus there is normally no requirement for purification or fractionation of the sample or isolation of analytes prior to performing the method.

In a further aspect, the invention relates to a construct for binding hydrophobic Coenzyme A ester comprising a heterologous peptide capable of binding at least one species of hydrophobic Coenzyme A ester, and a signal moiety.

Due to the high specificity and high affinity of these constructs towards hydrophobic CoA esters they are especially suited for use in the method for determination of the concentration of the hydrophobic compounds mentioned above.

The use of the constructs presented herein represents a unique way to measure free acyl-CoA concentrations of physiological important, highly amphiphatic, medium and long chain acyl-CoA esters. Long-chain acyl-CoA esters partition into membranes, stick to proteins and test tube walls. All previously published methods for measurement of acyl-CoA measure total acyl-CoA concentration including the very small fraction of free acyl-CoA, which is the biologically active fraction. This very small fraction can only be measured with the constructs according to the present invention. From the literature it is clear that knowledge of the free acyl-CoA concentration in vivo and in vitro conditions is the key to understand the function of these very important molecules in regulation of key cell functions including gene expression (Faergeman and Knudsen, 1997; Biochem J. 323, 1-12). One advantage with the present constructs is their high degree of specificity for hydrophobic-CoA esters only. The CoA head group determines the binding specificity of ACBP by interacting with specific amino acid residues in the binding site and contribute with 50% of the binding energy (Fægeman, et.al. 1996; Biochemistry, 35, 14118-26). ACBP does not bind fatty acids, nucleotide, prostaglandins and a number of other compound tested (Rosendal, et. al., 1993, Biochem J. 290,321-326). The high specificity makes the constructs very suitable for both in vitro and in vivo studies. The present work demonstrate the values of the constructs for in vitro determination of free acyl-CoA concentration. It is also envisaged and within the scope of the present invention to use the probes for in vivo studies.

The heterologous peptide preferably comprises a peptide having a high affinity for hydrophobic Coenzyme A esters, such as an acyl Coenzyme A binding protein or domain. Surprisingly, it has been found that the peptide conserves its high binding affinity for hydrophobic CoA esters even though the signal moiety is bound to the peptide. The signal moiety may even be bound to a carefully selected amino acid residue in the binding domain of the peptide, and still perform a high affinity binding to hydrophobic CoA esters.

The signal moiety preferably comprises an environmentally sensitive compound capable of emitting different signals in response to different environments. It is also surprising that the signal moiety retains its environmentally dependent signalling properties even though it is bound to a peptide according to the invention.

Through careful manipulation of the site for binding the signal moiety to the peptide, constructs may be modelled that are selective for one or for a group of species of hydrophobic CoA esters. Furthermore, through careful manipulation of the amino acid sequence in the peptide, especially in the binding domain of the peptide, constructs with a specific binding affinity for one species or for a group of species of hydrophobic CoA esters may be manufactured.

In a third aspect the invention relates to a kit for detection of the concentration of a hydrophobic Coenzyme A ester in a sample comprising at least a first construct according to the invention, and a sample compartment for application of the sample. The kits may be laid out in different ways for different applications and provide an easy and convenient way for performing the method for determination according to the invention without requirement for expensive and sophisticated equipment such as equipment for gas chromatography and often without any need for pre-treatment. Thus it is expected that the kits will be useful for performing assays in clinics for diagnosis, on farms for diagnosis of animal husbandry and/or for quality control of milk, in factories for quality control of lipid or fafty acid containing materials and/or products, for analysis of food, feed, blood, urine, milk, or other physiological fluids.

In yet another aspect, the invention relates to a nucleotide sequence encoding the heterologous peptide comprised in the construct according to the invention, an expression vector and a cell comprising this nucleotide sequence.

The heterologous peptide making up one part of the construct may conveniently be produced using recombinant molecular techniques.

According to a further aspect the invention relates to a method for determining the amount of free hydrophobic carboxylic acid(s) and/or lipid constituent(s) in a sample comprising

i. optionally fractionating the sample to obtain a substantially cell-free sample,

ii. mixing the substantially cell-free sample with an amount of water-miscible organic solvent to precipitate proteins and obtain a solution of free fatty acids,

iii. subjecting a sample of the supernatant to a quantitative analysis determining the amount of free fatty acids in the sample.

This method provides for easy and convenient extraction of free hydrophobic acids and lipids with the simultaneous precipitation of proteins that may interfere with the quantitative determination. The method is especially adapted for analysing blood samples using the hydrophobic CoA ester binding construct according to the present invention for quantitative determination.

### DEFINITIONS

Throughout the present application the term concentration is meant to include any concentration including 0. Thus it is an object of the present invention to measure the presence and or concentration of a given CoA ester or of CoA esters in a sample.

By specificity of a given construct with respect to a given CoA ester is meant specificity in the detected signal. This specificity may arise from a binding specificity but may also or additionally be caused by a signal specificity. Some constructs may thus bind a larger group of CoA esters but only produce a detectable signal in response to binding of one species or a group of CoA ester species. This is termed signal specificity.

The term ligand is used to designate a hydrophobic CoA ester capable of binding to a construct according to the invention. In a chemical sense the CoA ester may be regarded a ligand.

By “hydrophobic Co-enzyme A ester” is meant a Co-enzyme A ester, wherein the organic acid component of the acid is hydrophobic. In the sense of the present invention, the term also comprises CoASH as well as CoA esters of less hydrophobic carboxylic acids such as formic, acetic and butyric acid.

By free unbound CoA esters is meant the true free and unbound CoA esters. When the ester is first made from a free acid, it may not be unbound in a very strict sense, since it may be delivered to the binding construct directly from an acyl-Coenzyme A ligase. These CoA esters are also included in the term free unbound CoA esters for the purpose of the present invention.

By a signal is meant any signal detectable by detection techniques know to those skilled in the art. A signal—particularly a first signal within the meaning of the present invention—may also be 0.

The terms signal moiety and signal label are used interchangeably in the present application.

### FIGURES

FIG. 1. Alignment of 30 ACBP sequences (SEQ ID NO 1-30). The alignments are done to the bovine sequence with residues Ser 1 to Ile 86. The lengths of the other sequences are indicated as a subscript after the last residue shown and the four helices of bovine ACBP are shown as boxes above the sequences. Conserved Class 1 residues are present in 18 out of the 21 I- and b-ACBPs and are highlighted by black boxes. Conserved class 2 residues are hydrophobic residues (either M/L/H/P/A/F/Y/V/I) in all I- and b-ACBP sequences and in at least 27 out of all 30 sequences and are highlighted by grey boxes. Cysteines are in white text in grey boxes. Yeast(1) is from Saccharomyces cervisiae and Yeast(2) from Saccharomyces monoasensis and from Saccharomyces pastoranis (identical).

FIG. 2 shows a graphical description of measured fluorescence intensity in the 400 to 550 nm range resulting from titration with different levels of CoA, C4-CoA, C8-CoA C12-CoA, C16-CoA and C20-CoA with the badan derivative of M24C-bovine ACBP (Fluorescent modified Acyl-CoA Indicator 24 (FACI24)).

FIG. 3 shows a graphical description of measured fluorescence intensity in the 400 to 550 nm range resulting from titration with different levels of CoA, C4-CoA, C8-CoA C12-CoA, C16-CoA and C20-CoA with the badan derivative of A53C-bovine ACBP (Fluorescent Acyl-CoA Indicator 53 (FACI53)).

FIG. 4 shows isolelectrical point shift by bovine ACBP, M24C-bovineACBP and M24C-badan-bovine ACBP. Isoelectric focusing gels (PhastGel™ IEF 3-9) demonstrating the acyl-CoA binding profile of (a) r-bov ACBP, (b) A53C-badan and (c) M24C-badan. All the gels were prepared with ligands (1-7) in four-fold molar excess over protein. The band seen in lane 1 illustrates the unbound protein, which had a pl around 6 (confer with Broad pl Calibration Kit (pH 3-10)). The protein-ligand complex shifted to a pl around 3.8. Legend: 1: Water, 2: CoA-SH, 3: C4-CoA, 4: C8-CoA, 5: C12-CoA, 6: C16-CoA, 7: C20-CoA, 8: Broad pl Calibration Kit (pH 3-10).

FIG. 5 show quantitative determination of the concentration of total fatty acid in blood serum using FACI24 in combination with acyl-CoA synthetase. For experimental details see the text. A: Measurement of palmitoyl-CoA formed from palmitic acid bound to bovine serum albumin. FACI24 (3 μM) was incubated with the indicated amount of albumin bound Palmitic acid in the reaction mixture as described in the text. Excitation at 400 nm and emission reading at 470 nm. B: Measurement of total non-esterified free fatty acid in human serum. FACI24 (4 μM) was incubated with the indicated amounts of serum in the reaction mixture as described in the text. Excitation at 400 nm and emission reading at 470 nm.

FIG. 6 calculation of free acyl-CoA concentration in solutions of bovine ACBP titrated with different concentrations of palmitoyl-CoA In the presence of FACI24. For calculation details se the text.

FIG. 7 shows an overview of the different hydrophobic analytes that may be assayed according to the invention, together with appropriate pre-treatment steps.

FIG. 8 shows high performance Q-sepharose ion exchange separation M24C-bovine ACBP derivatives. The TCA precipitated protein was redissolved and loaded on a Q-sepharose HP column (1.5 cm×12 cm) equilibrated with 10 mM Tris-HCl pH 7.2 (buffer A). Proteins were eluted with a gradient of 0 to 400 mM NaCl (buffer B) as shown, with a flow of 3 ml/min. See Example 4 for further details. Solid line A₂₈₀, dashed line % buffer B.

FIG. 9 shows an analysis of FACI-24 by reverse phase HPLC. The desalted reaction product from synthesising Met24_Cys24-badan (FACI-24) was analysed on a24 Jupiter 5 μ, C18, 300A column equilibrated with 20% acetonitrile in water, 0.05% with trifluoroacetic acid (TFA) and eluted with a gradient to 80% acetonitrile in water, 0.05 % TFA as shown. The result shows that the product only contain one compound and that the A₂₈₀ and the A₃₈₇ absorbing material coelute. (Example 4).

FIG. 10 shows A: fluorescence emission spectra from titration of FACI-24 with C16:0-CoA. FACI-24 (3 μM) was titrated with C16:0-CoA as described in Example 6a. B: fluorescence emission spectra from titration of FACI-53 with C12-CoA and acyl-CoA esters. FACI-53 (3 μM) was titrated with CoA C12:0 as described in Example 6a. Exitation at 387 nm and emission at 460 nm. C: shows the change in fluorescence intensity when titrating FACI-24 (M24C-badan) with Acyl-CoA esters of different length. D: shows the change in fluorescence intensity when titrating FACI-53 (M53C-badan) with Acyl-CoA esters of different length.

FIG. 11 shows normalised fitted binding-curves for titration of FACI-24 with C8:0-C12:0- and C16:0-CoA. For experimental details see Example 6a. Exitation at 387 nm and emission at 460 nm.

FIG. 12 shows calculated relative increase in 460 nm emission upon addition of CoA and acyl-CoA esters to FACI-24. The columns represent average calculated 460 nm emission±sd from two independent experiments, at the chosen ligand/protein ratio, divided with 460 nm emission without ligand added.

FIG. 13 shows determination of GST-FadD activity using FACI-24 as a sensor for the rate of acyl-CoA formation. The reaction mixture (1.5 ml) contained 3 μM FACI-24 in 100 mM Tris-HCl pH 7.4, 1 mm DTT, 2 mM EDTA, 4 mM MgCl₂, 4 mM ATP, 60 μM CoA, 0.03 units/ml Acyl-CoA synthetase (GST-FadD), 3 μM BSA and 100 μM palmetic acid sodium salt. The reaction was followed by monitoring the increase in 460 nm emission (exitation 387 nm).

FIG. 14 shows determination of free fatty acids in an ethanol extraction of biological fluids. The reaction mixture (200 μl) contained 3 μM FACI-24 in 100 mM Tris-HCl pH 7.4, 1 mm DTT, 2 mM EDTA, 4 mM MgCl₂, 4 mM ATP, 60 μM CoA, 0.03 units/ml Acyl-CoA synthetase (GST-FadD) and 3 μM BSA and fatty acids as indicated added in 5 μl ethanol.

FIG. 15 shows a comparison of the results obtained with the present biding assay to results obtained with the NEFA C kit from WAKO Chemical Inc, Richmond, Va., USA.

### DETAILED DESCRIPTION OF THE INVENTION

The assay of this invention involves the single determination of signal intensity such as fluorescence intensity of signalling acyl CoA binding proteins (ACBP) such as fluorescent ACBP added to any aqueous solutions. The method directly determines the concentration of free acyl-CoA the activated form of fatty acids. If desired the method can determine the total fatty acid concentration in any biological solution when linked to acyl-CoA synthetase (ACS).

The principles and exemplary methods for constructing probes as described and defined herein and methods for measuring acyl-CoA levels is described in details below. Using these principles three different fluorescent-ACBP derivatives have been constructed. This has been done using three site directed mutated bovine ACBP (Met24_{—}24Cys, Ala53_{—}53Cys, and Met46_{—}46Cys) derivatised with badan (Molecular Probes). Two of these Met24_{—}24Cys (FACI24) and A53_{—}53Cys (FACI53) can serve as acylCoA probes, whereas Met46_{—}46Cys (FACI46) did not show changes in emission spectra with any of the tested ligands. To date the FACI24 is a preferable probe for acyl-CoA esters with from 14- to 20-carbons in the acyl-chains, with highest sensitivity to C16-CoA and FACI53 a preferable probe for C8- to C12 CoA esters. The repertoire of possible variants of the biosensor includes mutations of all the amino acid residues lining the binding cavity which include Phe49, Met24, Leu-25, Ala-53, Asp-21, Lys-50, Lys-54, Lys-18, pro-19, Ala-9, Tyr-31, Lys-32, Tyr-28, Tyr-73, Val-12, Lys-13, Leu-15; Ile-27; more preferably Met24, Ala53 and Lys50.

Neither FACI24 or FACI53 respond significantly to binding of free CoA which makes both sensors suitable for measuring acyl-CoA synthetase activity. Occupying, partly, the binding site by derivation of the mutated amino acid residue with a fluorescent group would be expected to partly perturb the acyl-CoA binding therefore altering the acyl-CoA binding constant. Surprisingly the replacement of the —CH₂-S-CH₃ part Met-24 with the badan group increased the binding affinity. The K_{D} C14-CoA binding as determined by isothermal titration microcalorimetry (Færgemann etl al, Biochemistry 1996, 35:14118-14126) and titration equilibrium analysis (Table 2) in >0.1 M salt is 16 nM and 1.7 nM for native bovine ACBP and FACI24 respectively. However as long as the derivatised molecule can bind the acyl-CoA ester it can still function as an acyl-CoA probe. As long as its dynamic range is sufficient, the acyl-CoA and CoA levels, over a wide range, including those that are physiological, can be measured. This range can be further broadened by introducing additional mutations (Kragelund et al, 1999, Biochemistry 38 (8) pp 2386-94) or by in deleting or inserting one or more amino acid residues as seen in Plasmodium falciparum ACBP (unpublished data).

A costruct is prepared by any of the techniques describe below, or other techniques that can, using the guidance of this disclosure, be adapted to such a preparation. The construct comprises a heterologous peptide that has been labelled with a signal moiety that, when so labelled specifically binds hydrophobic-CoA esters and exhibits one signal when unbound and a measurably different signal when bound to hydrophobic-CoA esters and the signal difference being detectable. Native acyl-CoA binding proteins (ACBP) or mutated ACBP can be used to provide CoA and hydrophobic-CoA ester reactive binding sites.

The Heterologous Peptide

Acyl Coenzyme A Binding Protein (ACBP)

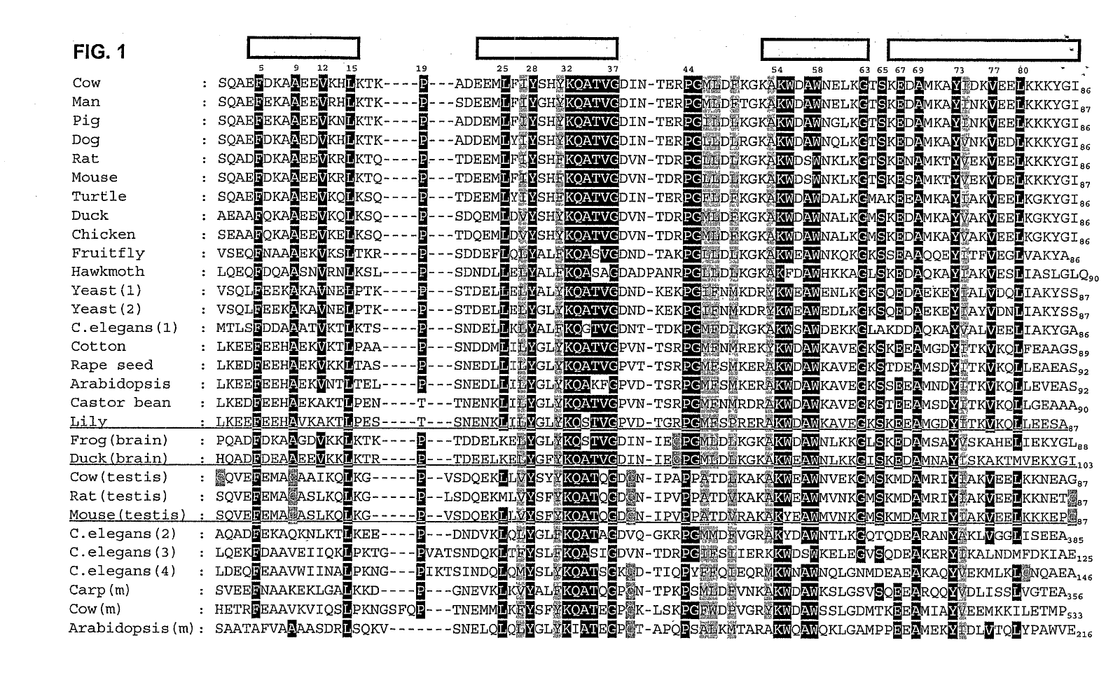

The heterologous peptide comprised in the construct according to the invention preferably comprises an acyl-CoenzymeA binding protein, a variant or functional equivalent thereof. Acyl-Coenzyme A binding proteins (ACBPs) are known in the art from a wide variety of species including animals, plants and lower organisms. Wild-type ACBP is an 86-103 residue protein with a highly conserved amino acid sequence. It has been isolated from a wide range of species ranging from yeasts and plants to reptiles and man, but also several proteins translated from gene sequences, especially from Caenorhabditis elegans, have been suggested. A total of 30 sequences are disclosed in FIG. 1.

From the alignment, at least four groups of ACBP can be identified. The first group is the generally expressed ACBP isoform, first isolated from bovine liver (l-ACBP, SEQ-ID NO 30). In their wild type form these ACBPs contain no cysteines and are 86-92 residues long. The second group is the testis specific isoform (t-ACBP) also called endozepine-like protein (ELP). T-ACBPs have now been isolated from three different species and these three all wild-type t-ACBPs contain three cysteines. A putative third group may be a brain specific isoform of ACBP (b-ACBP) which has been deduced from gene sequences from duck and frog brain and which contain in their wild type form one single cysteine at position 43. The fourth group of native ACBP is a group of longer sequences with up to 533 amino acids. Some of these longer sequences are suggested to be membrane bound isoforms (m-ACBP), whereas others remain to be isolated as proteins. Many of these longer forms comprise cysteine(s).

The construct according to the invention preferably comprises an acyl-Coenzyme A binding protein such as an acyl-CoA binding protein comprising an amino acid sequence from the sequences of FIG. 1 (SEQ-ID NO 1-30) a variant or functional equivalent thereof.

Using the sequences (SEQ ID NO 1-30) the skilled protein chemist may easily identify homologous proteins in other species and even novel proteins having essentially the same affinity for CoA esters of hydrophobic acids. All these proteins and their functional variants are within the scope of the present invention.

The heterologous peptide of the construct may also preferably comprise an acyl-CoenzymeA binding domain. This domain could be isolated from a larger protein such as those shown in FIG. 1 (SEQ ID NO 1, 4, 5, 6, 7, 11) or from homologous proteins from those and other species.

According to a preferred embodiment of the invention the heterologous peptide comprises a modified form of bovine ACBP (SEQ ID NO 30), a variant or functional equivalent thereof. A number of constructs have been produced based on bovine ACBP and have shown to work well under laboratory conditions.

The Linkage Between the Peptide and Signal Label

The signal moiety or signal label may be bound to the heterologous peptide via a cystein residue for binding the signal moiety. This cystein could be natively present in the construct or be introduced via substitution or addition.

Another possibility is that signal label is bound to a lysine residue, which likewise may be present in a native peptide or introduced by substitution or addition of an amino acid residue.

Methods are well known in the art for binding compounds having specific groups to the side chains of cystein and lysin residues. However, it also lies within the scope of the present invention to link the signal moiety to the side chain of any other amino acid residue in the presence of a suitable and specific reaction. Such reaction may comprise but is not limited to nucleophilic substitution or addition, or electrophilic substitution or addition reaction, esterification, thioesterification, condensation reactions, amide reactions. Preferably the reaction is a specific reaction, so that the number and the position of signal moieties linked to the peptide is closely controlled. Such other amino acid residues include but are not limited to trp, ser, thr, tyr, asp, glu, his. Preferably, the linkage should be performed without substantially altering the signalling properties of the signal moiety.

Preferably the heterologous peptide comprises only one residue of the type to which the signal moiety is to be linked. In the presence of two or more residues of the same type such as two or more cysteines, a signal moiety may be bound to both of the cystein residues.

If more than one signal moiety is to be linked to the construct and if these more than one signal moieties are different, they may advantageously be linked to different amino acid residues in order to facilitate the specificity of the linkage.

The amino acid residue, to which the signal label is bound may be selected from the amino acid residues aligning the acyl Coenzyme A binding domain. The residue may also be selected from the amino acid residues having van der Waals' contact with a bound hydrophobic Coenzyme A ester or it may be selected from the amino acid residues being within 5 Å from a bound hydrophobic Coenzyme A ester.

The residue may likewise be selected from the amino acid residues making up the α-helices of the heterologous peptide.

More specifically the heterologous peptide may comprise the bovine ACBP (SEQ ID NO 30) and the native amino acid being replaced by a cystein residue is preferably selected from the group consisting of Phe-49, Met24, Leu-25, Ala-53, Asp-21, Lys-50, Lys-54, Lys-18, pro-19, Ala-9, Tyr-31, Lys-32, Tyr-28, Tyr-73, Val-12, Lys-13, Leu-15, Ile-27. More preferably the amino acid being substituted by a cystein residue is selected from the group consisting of Met-24, Ala-53, and Lys-50.

The position of the amino acid residue carrying the signal label may determine the specificity of the construct with respect to the hydrophobic CoA esters. Through careful selection of the residue carrying the signal label, constructs being specific for a specific hydrophobic CoA ester or being specific for a group of hydrophobic CoA esters may be designed.

However, other changes, such as substitution, deletion or addition, to the amino acid sequence of the heterologous peptide may also affect the binding properties of the peptide in the sense that two constructs having the signal moiety bound to the same amino acid residue but differing at another position, may have different binding affinity towards a hydrophobic CoA ester. Similarly two such different constructs may bind the same CoA esters but exhibit different signals in response to binding different CoA esters.

More specifically, in the above mentioned case, where Met 24 is mutagenised to cysteine and a Badan moiety is bound to this cysteine, the binding affinity can be changed by substituting Lys32 with alanine, arginine, or glutamine. Thereby acyl-CoA binding constructs having a K_{D} ranging from 0.5 nM to 1500 nM can be obtained.

Variants

The amino acid sequence of the heterologous peptide preferably has at least 30% sequence identity to one of the sequences (SEQ ID NO 1-30) of FIG. 1, such as at least 40% sequence identity, for example at least 50% sequence identity, such as at least 55% sequence identify, for example at least 60% sequence identity, such as at least 65% sequence identity, for example at least 70% sequence identity, such as at least 75% sequence identity, for example at least 80% sequence identity, such as at least 85% sequence identity, for example at least 90 % sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity.

A variant of the sequences of FIG. 1 (SEQ ID NO 1-30) according to the invention may appropriately be defined with reference to the four α-helices, which the variants preferably comprise. These are in the following termed A1, A2, A3 and A4. These four helices are preferably linked together by an A1-A2 linking peptide, an A2-A3 linking peptide, and an A3-A4 linking peptide. The variants preferably also comprise a N-terminal peptide and a C-terminal peptide.

Preferred variants are in the following described with reference to these 8 constituents.

A1 preferably comprises 12 amino acids capable of forming an α-helix, which may be described by the general formula: X1-X-X-X-2-X-X-3-X-X-4, where X denotes any individually selected amino acid; 1 preferably denotes a leu but may also denote ala; 2 preferably denotes ala but also may denote cys, gln, val, or lys; 3 preferably denotes val but also may denote ala, ile, leu, or ser; and 4 preferably denotes leu.

A2 preferably comprises 16 amino acids capable of forming an α-helix, which may be described by the general formula: X-X-X-1-X-2-3-X-X-4-5-6-7-8-9-10, where X denotes any individually selected amino acid. 1 preferably denotes leu, but also may denote K or M; 2 preferably denotes a hydrophobic residue, more preferably an ile residue, but it may also denote a val, a leu, a phe or met residue; 3 preferably denotes a tyr residue; 4 preferably denotes a hydrophobic residue, more preferably a tyr or phe residue; 5 preferably denotes a lys residue; 6 preferably denotes a gln residue but also may denote an ile residue; 7 preferably denotes a ala residue, but also may denote a gly or ser residue; 8 preferably denotes a thr residue, but also may denote a ser or lys residue; 9 preferably denotes a val residue but also may denote an ala, phe, gln, ala, ile, ser or glu residue; 10 preferably denotes a gly residue.

A3 preferably comprises 12 amino acids capable of forming an α-helix, which may be described by the general formula: X-1-2-3-X-4-5-X-X-X-X-6, where X denotes any individually selected amino acid; 1 preferably denotes a hydrophobic amino acid residue more preferably an ala residue, but it may also denote a tyr, a lys or a met residue; 2 preferably denotes a lys residue; 3 preferably denotes a trp residue, but it may also denote a phe or a tyr residue; 4 preferably denotes an ala residue but may also denote a ser residue; 5 preferably denotes a trp residue; and 6 preferably denotes a gly residue, but may also denote an asn, a ser, an asp, or an ala residue.

A4 preferably comprises 20 amino acids capable of forming an α-helix, which may be described by the general formula: X-1-X-2-X-X-X-3-4-X-X-5-X-X-6-X-X-X-X-X, where X denotes any individually selected amino acid; 1 preferably denotes a glu residue but may also denote an asp or a met; 2 preferably denotes an ala residue; 3 preferably denotes a tyr residue; 4 preferably denotes a hydrophobic residue, more preferably an ile, a val, or an ala residue; 5 preferably denotes a val residue, but may also denote an ala, a leu, a met or an ile residue; 6 preferably denotes a leu residue, but may also denote a met or an ile residue.

The A1-A2 linking peptide preferably comprises from 6 to 10 amino acid residues. When the A1-A2 linking peptide consists of 6 amino acids, amino acid residue number 3 or 4 preferably is a pro residue. When it consists of 10 amino acid residues, amino acid number 5 or 8 preferably is a pro residue.

The A2-A3 linking peptide preferably comprises 14 to 15 amino acid residues capable of forming an overhand loop which may be described by the general formula: X-1-X-2-X-X-X-3-4-5-6-X-7-X-X, wherein X denotes any individually selected amino acid residue. 1 may denote a cystein residue. 2 preferably denotes no amino acid resulting in a peptide of 14 residues, however when present it preferably denotes a pro residue. 3 preferably denotes a pro residue. 4 preferably denotes a gyl residue, but it may also denote a pro residue, a tyr residue or a ser residue. 5 preferably denotes a hydrophobic residue, more preferably a met residue, a leu residue, a phe residue, an ile residue or an ala residue. 6 preferably denotes a hydrophobic residue, more preferably a leu residue, a phe residue, a met residue or a trp residue. However 6 may also denote a thr residue. 7 preferably denotes a hydrophobic residue, more preferably a phe residue, a leu residue, a met residue, a pro residue, a val residue or an ile residue.

The A3-A4 linking peptide preferably comprises 2 amino acids, having the general formula X-1, wherein X denotes any individually selected amino acid, and 1 preferably denotes a ser residue, but it may also denote an ala residue, a thr residue, an asp residue, or a pro residue.

Variants of the sequences in FIG. 1 may also comprise a C-terminal peptide and/or a N-terminal peptide. Full length proteins corresponding to sequences in FIG. 1 thus further comprise N terminal peptides of 3, 24 and 41 amino acids, and C terminal peptides of 19, 33, 52, 117, 276, 327, and 403 amino acids. Thus it is conceivable to the skilled person that the length of the peptide may be much longer than the length of the acyl-CoA binding domain displayed in FIG. 1 without substantially altering the binding capability of the peptide. A specific type of peptide that may be added to a terminal, preferably to the N-terminal end of a peptide according to the invention is an affinity tag, such as a His tag or a GST tag. Experiments have shown that it is possible to add a poly His tag comprising e.g. 6 His residues and a linker residue without substantially altering the binding capabilities of the peptide. It is thus not necessary to cleave off the poly His tail after purification.

The peptide may furthermore comprise a proteinase cleavage site for cleaving off a tag, which is only used during purification of the peptide.

It is expected that by making substitutions, deletions and/or insertions of amino acid residues, the specificity of the heterologous peptide with respect to CoA esters is changed and/or the signal emitted or detected is changed.

Accordingly, a variant of the sequences in FIG. 1 or fragments thereof according to the invention may comprise, within the same variant of the sequences in FIG. 1 or fragments thereof or among different variants of the sequences in FIG. 1 or fragments thereof, at least one substitution, such as a plurality of substitutions introduced independently of one another. Variants of the sequences in FIG. 1 or fragments thereof may thus comprise conservative substitutions independently of one another, wherein at least one glycine (Gly) of said variants of the sequences in FIG. 1 or fragments thereof of the sequences in FIG. 1 is substituted with an amino acid selected from the group of amino acids consisting of Ala, Val, Leu, and Ile, and independently thereof, variants of the sequences in FIG. 1 or fragments thereof, wherein at least one of said alanines (Ala) of said variant of the sequences in FIG. 1 or fragments thereof is substituted with an amino acid selected from the group of amino acids consisting of Gly, Val, Leu, and lie, and independently thereof, variants of the sequences in FIG. 1 or fragments thereof, wherein at least one valine (Val) of said variants of the sequences in FIG. 1 or fragments thereof is substituted with an amino acid selected from the group of amino acids consisting of Gly, Ala, Leu, and Ile, and independently thereof, variants of the sequences in FIG. 1 or fragments thereof, wherein at least one of said leucines (Leu) of said variant of the sequences in FIG. 1 or fragments thereof is substituted with an amino acid selected from the group of amino acids consisting of Gly, Ala, Val, and Ile, and independently thereof, variants of the sequences in FIG. 1 or fragments thereof, wherein at least one isoleucine (Ile) of said variants of the sequences in FIG. 1 or fragments thereof is substituted with an amino acid selected from the group of amino acids consisting of Gly, Ala, Val and Leu, and independently thereof, variants of the sequences in FIG. 1 or fragments thereof wherein at least one of said aspartic acids (Asp) of said variants of the sequences in FIG. 1 or fragments thereof is substituted with an amino acid selected from the group of amino acids consisting of Glu, Asn, and Gln, and independently thereof, variants of the sequences in FIG. 1 or fragments thereof, wherein at least one of said phenylalanines (Phe) of said variants of the sequences in FIG. 1 or fragments thereof is substituted with an amino acid selected from the group of amino acids consisting of Tyr, Trp, His, Pro, and preferably selected from the group of amino acids consisting of Tyr and Trp, and independently thereof, variants of the sequences in FIG. 1 or fragments thereof, wherein at least one of said tyrosines (Tyr) of said variants of the sequences in FIG. 1 or fragments thereof is substituted with an amino acid selected from the group of amino acids consisting of Phe, Trp, His, Pro, preferably an amino acid selected from the group of amino acids consisting of Phe and Trp, and independently thereof, variants of the sequences in FIG. 1 or fragments thereof, wherein at least one of said arginines (Arg) of said fragment of the sequences in FIG. 1 is substituted with an amino acid selected from the group of amino acids consisting of Lys and His, and independently thereof, variants of the sequences in FIG. 1 or fragments thereof, wherein at least one lysine (Lys) of said variants of the sequences in FIG. 1 or fragments thereof is substituted with an amino acid selected from the group of amino acids consisting of Arg and His, and independently thereof, variants of the sequences in FIG. 1 or fragments thereof, wherein at least one of said aspargines (Asn) of said variants of the sequences in FIG. 1 or fragments thereof is substituted with an amino acid selected from the group of amino acids consisting of Asp, Glu, and GIn, and independently thereof, variants of the sequences in FIG. 1 or fragments thereof, wherein at least one glutamine (Gln) of said variants of the sequences in FIG. 1 or fragments thereof is substituted with an amino acid selected from the group of amino acids consisting of Asp, Glu, and Asn, and independently thereof, variants of the sequences in FIG. 1 or fragments thereof, wherein at least one proline (Pro) of said variants of the sequences in FIG. 1 or fragments thereof is substituted with an amino acid selected from the group of amino acids consisting of Phe, Tyr, Trp, and His, and independently thereof, variants of the sequences in FIG. 1 or fragments thereof, wherein at least one of said cysteines (Cys) of said variants of the sequences in FIG. 1 or fragments thereof is substituted with an amino acid selected from the group of amino acids consisting of Asp, Glu, Lys, Arg, His, Asn, Gln, Ser, Thr, and Tyr.

It is clear from the above outline that the same variant or fragment thereof may comprise more than one conservative amino acid substitution from more than one group of conservative amino acids as defined herein above.

The addition or deletion of an amino acid may be an addition or deletion of from 2 to 10 amino acids, such as from 10 to 20 amino acids, for example from 20 to 30 amino acids, such as from 40 to 50 amino acids. However, additions or deletions of more than 50 amino acids, such as additions from 10 to 100 amino acids, addition of 100 to 150 amino acids, addition of 150-250 amino acids, are also comprised within the present invention.

It will thus be understood that the invention concerns a heterologous peptide comprising at least one fragment of the sequences in FIG. 1 capable of binding at least one species of hydrophobic CoA esters, including any variants and functional equivalents of such at least one fragment.

The heterologous peptide according to the present invention, including any functional equivalents and fragments thereof, may in one embodiment comprise less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues.

Fragments

A fragment comprising the acyl-CoA binding region of the native sequences in FIG. 1 is particularly preferred. However, the invention is not limited to fragments comprising the acyl-CoA binding region. Deletions of such fragments generating functionally equivalent fragments of the sequences in FIG. 1 comprising less than the acyl-CoA binding domain are also comprised in the present invention. Functional equivalents of the sequences in FIG. 1 peptides, and fragments thereof according to the present invention, may comprise less or more amino acid residues than the acyl-CoA binding region.

“Functional equivalency” as used in the present invention is according to one preferred embodiment established by means of reference to the corresponding functionality of a predetermined fragment of the sequences in FIG. 1. More specifically, functional equivalency is to be understood as the ability of the functional equivalent to bind specifically to CoA esters of hydrophobic acids or to at least one species of CoA esters of hydrophobic acids. By specific binding is meant that the K_{D} of the complex between the CoA ester and the heterologous peptide is below 2 μM, such as below 1.5 μM, for example below 1.0 μM, preferably below 500 nM, more preferably below 200 nM such as below 100 nM, for example below 90 nM, such as below 80 nM, for example below 70 nM, such as below 60 nM, for example below 50 nM, such as below 40 nM, for example below 30 nM, such as below 20 nM, for example below 15 nM, such as below 10 nM, for example below 8 nM, such as below 7 nM, for example below 6 nM, such as below 5 nM, for example below 4 nM, such as below 3 nM, for example below 2 nM, such as below 1 nM.

Functional equivalents of variants of the sequences in FIG. 1 will be understood to exhibit amino acid sequences gradually differing from the preferred predetermined sequence, as the number and scope of insertions, deletions and substitutions including conservative substitutions increases. This difference is measured as a reduction in homology between the preferred predetermined sequence and the fragment or functional equivalent.

All fragments or functional equivalents of ACBPs are included within the scope of this invention, regardless of the degree of homology that they show to a preferred predetermined sequence of ACBP. The reason for this is that some regions of the sequences in FIG. 1 are most likely readily mutatable, or capable of being completely deleted, without any significant effect on the binding activity of the resulting fragment.

A functional variant obtained by substitution may well exhibit some form or degree of native activity of the sequences in FIG. 1, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity between i) a given the sequences in FIG. 1 fragment capable of effect and ii) a preferred predetermined fragment, is not a principal measure of the fragment as a variant or functional equivalent of a preferred predetermined the sequences in FIG. 1 fragment according to the present invention.

The homology between amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90.

Fragments sharing at least some homology with the sequences in FIG. 1 fragment are to be considered as falling within the scope of the present invention when they are at least about 40 percent homologous with the ACBP or fragment thereof, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the sequences in FIG. 1 or fragments thereof. According to one embodiment of the invention the homology percentages refer to identity percentages.

Additional factors that may be taken into consideration when determining functional equivalence according to the meaning used herein are i) the ability of antisera raised against the peptides of FIG. 1 to detect a fragment of the sequences in FIG. 1 according to the present invention, or ii) the ability of the functionally equivalent fragment to compete with the sequences in FIG. 1 in a CoA ester binding assay.

Conservative substitutions may be introduced in any position of a preferred predetermined ACBP peptide or fragment thereof. It may however also be desirable to introduce non-conservative substitutions, particularly, but not limited to, a non-conservative substitution in any one or more positions.

A non-conservative substitution leading to the formation of a functionally equivalent fragment of the sequences in FIG. 1 would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the variants described herein, sterically similar variants may be formulated to mimic the key portions of the variant structure and that such compounds may also be used in the same manner as the variants of the invention. This may be achieved by techniques of modelling and chemical designing known to those of skill in the art. It will be understood that all such sterically similar constructs fall within the scope of the present invention.

Signal Labels

The label, which is linked to the heterologous peptide according to the invention, may be termed a signal moiety or a signal label. In the following these terms are used interchangably.

The signal label is preferably linked to the heterologous peptide via a covalent linkage. Such a linkage could be made e.g. between the label and a cystein or a lysin residue in the peptide. The signal label is preferably of the type that changes its signal in response to a change in the environment and/or conformation, e.g. a change in the polarity of the environment. The signal label may thus comprise a fluorescent label, a chromogenic label, a chemoluminescent label, or a photoluminescent label.

Exemplary fluorescent labels are described below. The nature of the fluorescent label is not critical however, it need only to be capable of being attached to the specific heterologous peptide and, when attached emit fluorescence measurably different when the protein is bound with a CoA ester compared to the fluorescence emitted when unbound. The mode of detection is also not critical. In other words, the label and the mode of detection are not critical limiting factors in this invention.

The fluorescent moiety preferably comprises a compound selected from the group consisting of acrylodan; 5-dimethylaminonaphtalene-1-sulfonyl aziridine (danzyl aziridine); 4-[N-[2-iodoacetoxy)ethyl]-N-methylamino]-7-nitrobenz-2-oxa 1,3 diazole ester (IANBDE); 4-[N-[2-iodoacetoxy)ethyl]-N-methylamino]-7-nitrobenz-2-oxa 1,3 diazole amide (IANBDA); 6-acryloyl-2-dimtheylaminonaphtalene (acrylodan); N-(7-chlorobenz-2-oxa-1,3-diazyl-4-yl)sulfonyl morpholine; 4-chloro-7-nitrobenz-2-oxa-1,3-diazole (NBD chloride); didansyl-L-cystine; N,N′-dimethyl-N-(iodoacetyl)-N′-(7-nitrobenz-2-oxa-1,3-diazol-4-yl)ethylenediamine (IANBD amide); 7-fluorobenz-2-oxa-1,3-diazole-4-sulfonamide (ABD-F); 4-fluoro-7-nitrobenz-2-oxa-1,3-diazole (NBD fluoride); 2-(4′-(iodoacetamido)anilino)naphtalene-6-sulfonic acid, sodium salt (IAANS); 5-(((2-iodoacetyl)amino)ethyl)amino)naphtalene-1-sulfonic acid (1,5-IAEDANS); 2-(4′-maleimidylanilino)naphtalene-6-sulfonic acid (MIANS); N-(1-pyreneethyl)iodoacetamide; N-(1-pyrene)iodoacetamide; N-(1-pyrene)maleimide; N-(1-pyrenemethyl)iodoacetamide (PMIA amide); 1-pyrenemethyl iodoacetate (PMIA ester); N-(1-pyrenepropyl)iodoacetamide); 1-(2,3-epoxypropyl)-4-(5-(4-methoxyphenyl)oxazol-2-yl)pyridinium trifluoromethanesulfonate (PyMPO epoxide); erythrosin-5-iodoacetamide; fluorescein-5-maleimide; 5-iodoacetamidofluorescein (5-IAF); 6-iodoacetamidofluorescein (6-IAF); 1-(2-maleimidylethyl)-4-(5-(4-methoxyphenyl)oxazol-2-yl)pyridinium methanesulfonate (PyMPO maleimide); Oregon Green™ 488 iodoacetamide “mixed isomers”; tetramethylrhodamine-5-iodoacetamide (5-TMRIA) “single isomer”; tetramethylrhodamine-5-maleimide “single isomer”; tetramethylrhodamine-6-maleimide “single isomer”; Texas Red® C₅ bromoacetamide; Texas Red® C₂ maleimide. More preferably the fluorescent moiety comprises Badan.

The fluorescent moiety may also comprise derivatives of the compounds mentioned above.

Furthermore, the construct may comprise a linker molecule for linking the fluorescent moiety with the peptide. The role of the linker molecule may be to facilitate the chemical bonding of the signal moiety to an amino acid residue in the peptide or the role may be to position the spacer moiety in relation to the peptide.

The construct may also further comprise a second signal moiety. The second signal moiety may similarly comprise a fluorescent label, a chromogenic label, a chemoluminescent label, or a photoluminescent label. Preferably the second signal moiety comprises a compound selected from the group of fluorescent labels listed above. The first and second signal moiety may comprise the same compound or they may preferably comprise two different compounds.

The effect of binding a second signal label to the heterologous peptide may be to change the specificity of the construct vis a vis the ligand and/or to affect the signal change upon binding of the ligand. By having e.g. two flourescent labels attached to a heterologous peptide according to the invention, it may be possible not only to obtain an increase in the emission at one wavelength but in addition a simultaneous decrease in the emission at another wavelength compared to unbound construct. Thereby a more precise signal can be recorded. The inventors also envisage that binding of different ligands to a construct comprising two or more signal labels will result in differential change in the emission at two different wavelengths, thereby allowing identification of the ligand bound to the construct through a mathematical combination of emission change at two or more different wavelengths. The second ligand is preferably bound to an amino acid positioned, so that the ligand is moved from a hydrophobic to a hydrophilic environment upon binding of the hydrophobic CoA ester.

The difference in fluorescence between a solution comprising a construct according to the invention and the solution comprising a construct-hydrophobic CoA ester complex is detected or measured. The change in fluorescence is related to the amount of free hydrophobic-CoA esters in the solution. This may be a qualitative relationship i.e., hydrophobic-CoA ester present or not present above some threshold level, but in most instances the fluorescence change is related quantitatively to the concentration of hydrophobic CoA ester. Once the hydrophobic-CoA ester dissociation constant (K_{D}) has been determined, the concentration of the hydrophobic CoA ester can be calculated from the detected signal.

The Signal

The detected signal according to the invention may comprise a fluorescence signal, a chromogenic signal, a chemiluminiscense signal, or a photoluminiscense signal.

The detected signal may comprise the second signal (which is the signal detectable after binding of the CoA ester to the construct). In this case the first signal preferably is essentially zero so that the difference between the signals do not have to be calculated. Alternatively, the detected signal may comprise the difference between the first and the second signal or the detected signal may comprise a mathematical combination between two different signals such as between two signals emitted or detected at two different wavelengths.

Preferably, the detected signal is essentially proportional to the amount of hydrophobic Coenzyme A ester in the sample such as being essentially proportional to the amount of at least one species of Coenzyme A ester in the sample.

According to an especially preferred embodiment of the invention, the at least one species of Coenzyme A ester for which the detected signal is essentially proportional to its amount comprises a species selected from the group consisting of Coenzyme A esters with a C2 acyl group, a C4 acyl group, a C6 acyl group, a C8 acyl group a C10 acyl group, a C12 acyl group, a C14 acyl group, a C16 acyl group, a C18 acyl group, a C20 acyl group, a C22 acyl group, a C24 acyl group, a C26 acyl group, a saturated acyl group, a mono-unsaturated acyl group, a polyunsaturated acyl group, an acyl group comprising a cis double bond, an acyl group comprising a trans double bond, an acyl group comprising a ring structure, an acyl group comprising a side chain.

Thereby it is possible to selectively detect the amount of one species or a group of species, which are related in terms of similar length or similar configurations in the side chain.

Similarly, the detected signal from a first species of hydrophobic Coenzyme A ester may be essentially 0 (i.e. no binding to the construct) and the detected signal from a second species of hydrophobic Coenzyme A may be essentially proportional to the amount of said second species in the sample (binding to the construct and thus signal).

The first species may comprise a saturated species and the second species may comprise an unsaturated species or vice versa. The first species may comprises a mono-unsaturated species and the second species a poly-unsaturated species or vice versa. The first species may comprise a species with a cis-double bond and the second species a trans-double bond or vice versa. The first species may comprise a double bond and the second species comprises a double bond in another position.

According to another embodiment of the invention the detected signal may be essentially proportional to the amount of a group of hydrophobic Coenzyme A esters in the sample. This group may comprise Coenzyme A esters with a C2-C6 acyl group, Coenzyme A esters with a C8-C12 acyl group, Coenzyme A esters with a C12-C16 acyl group, Coenzyme A esters with a C16-C20 acyl group, Coenzyme A esters with a C12-C20 acyl group, Coenzyme A esters with a C22-C24 group, Coenzyme A esters with a C6-C10 acyl group, or a C10-C14 acyl group, or a C14-C18 acyl gruop, or a C18-C22 acyl group, or a C4-C8 acyl group, or a C8-C16 acyl group, or a C4-C12 acyl group, or an acyl group comprising more than 20 carbon atoms. Thus it is envisaged that it is possible to design a construct according to the invention which is specific for any one group of CoA esters having some chemical property in common. Through use of several constructs having specificity for different groups of species, differential analysis of a complex sample may be performed.

By careful design of two or more constructs each being specific for a different group of CoA species and each providing a measurably different signal when bound it may be possible to detect in one step the concentration of more than one group of species of CoA in a single sample. In order to make full use of this option, the two or more constructs should have a specific binding affinity of the different groups of CoA species and they should also measurably different signals upon binding of the CoA species.

Dissociation Constant

As stated above, the dissociation constant, K_{D}, of the complex between C14-CoA and native ACBP is 16 nM, which indicates a very strong binding between the protein and the ligand. The K_{D} between hydrophobic CoA esters and the constructs according to the invention preferably is below below 2 μM, such as below 1.5 μM, such as below 1.0 μM preferably below 500 nM, more preferably below 200 nM, such as below 100 nM, for example below 90 nM, such as below 80 nM, for example below 70 nM, such as below 60 nM, for example below 50 nM, such as below 40 nM, for example below 30 nM, such as below 20 nM, for example below 15 nM, such as below 10 nM, for example below 8 nM, such as below 7 nM, for example below 6 nM, such as below 5 nM, for example below 4 nM, such as below 3 nM, for example below 2 nM, such as below 1 nM, for example below 0.5 nM, such as below 0.1 nM.

The K_{D} of the construct according to the invention may be determined with reference to one species of CoA ester, to a group of CoA esters or to CoA esters in general. In order to be able to detect specific species or groups of species of CoA esters the K_{D} with respect to this one species or group of species preferably is lower than the K_{D} of the same construct with respect to other CoA esters. By lower is preferably meant at least 10 times lower, more preferably at least 100 times lower.

Specificity of Signal

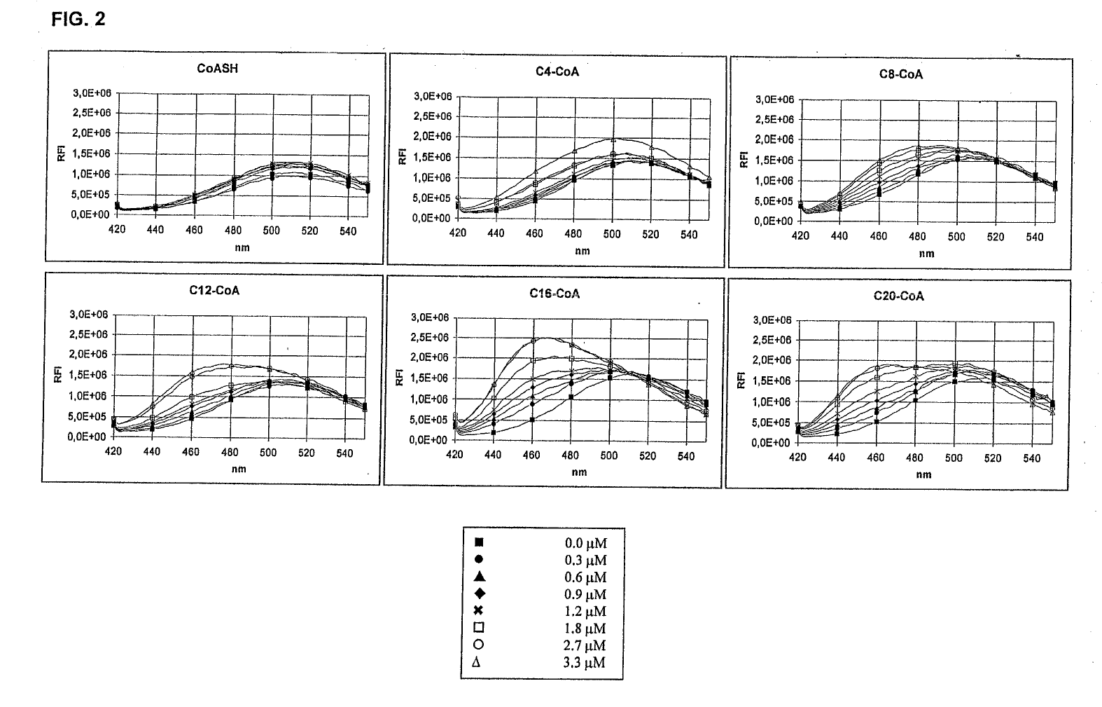

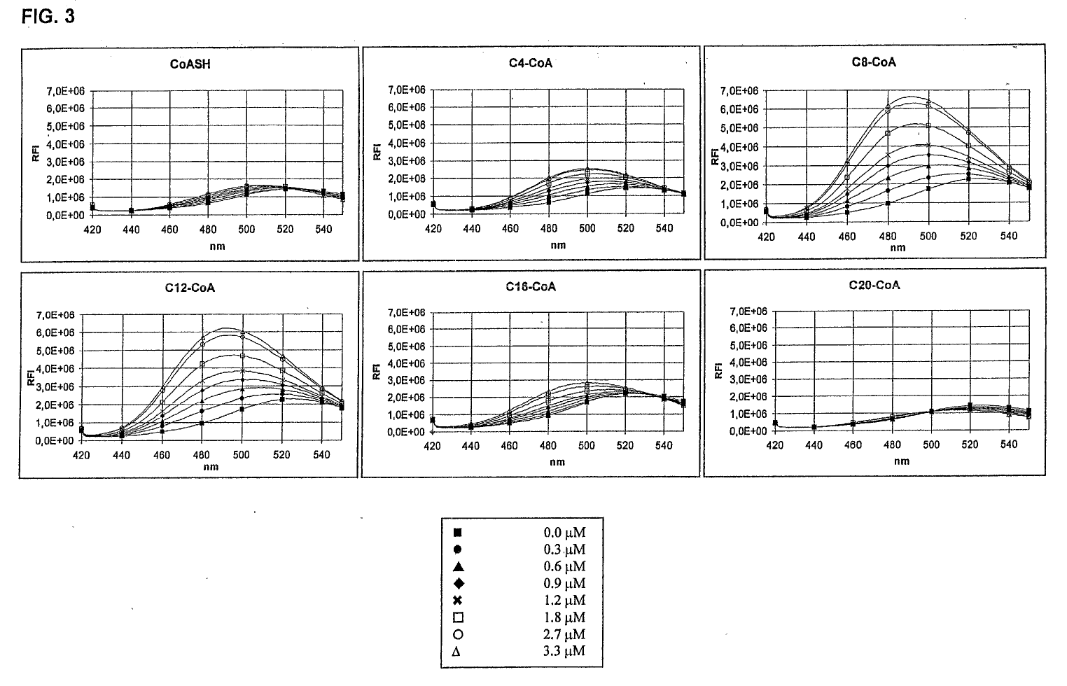

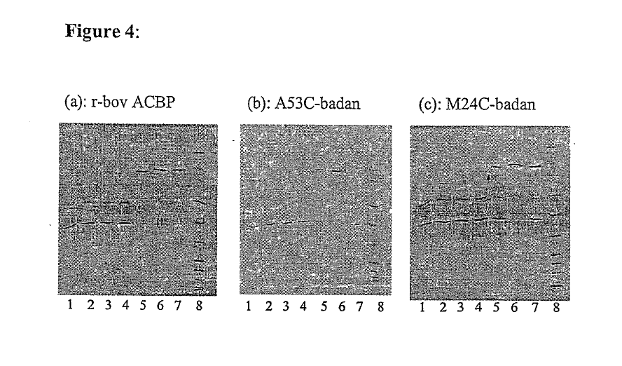

Reference is made to FIG. 2. and FIG. 3. which depict the emission spectra of FACI24 and FACI53 titrated with increasing concentrations of CoA, C4-, C8-, C12-, C16- and C20-CoA esters. The addition of ligand and measurement of emission of FACI24 and FACI53 was performed as described in example 2. The increased addition of ligand to the mutated and modified proteins caused a proportional spectral change. The normal physiological binding profile was confirmed by direct binding studies using isoelectrical focusing as predicted in FIG. 4. This demonstrates that mutation and fluorescent modification does not abolish the acyl-CoA binding characteristics of bovine ACBP. In fact FACI24 binds C14-CoA with higher affinity (K_{D}=1.7 nM, Table 2) than native bovine ACBP (K_{D} 16 nM). The results clearly indicate that fluorescence emission at 470 nm may provide a measure for the concentration of free unbound long-chain(>C12)-acyl-CoA ester. The emission profile of FACI53 differed from that of FACI24 in that emission maximum was observed at 487 nm instead of 465 nm and that FACI53 exhibits highest sensitivity for C8- to C12-acyl-CoA and the probe hardly responded to CoA and C20-CoA binding. The lack of fluorescence response to C16-CoA and C20-CoA was not due to lack of binding, both acyl-CoA were shown to bind to FACI53 by isoelectrical focusing (FIG. 4) and C16-CoA binding was confirmed by isothermal titration calorimetry.

These results clearly demonstrate that acyl-CoA sensor probes can be designed by engineering of the binding site at different locations with flourescent groups sensitive to differences in the environment. The two sensors presented herein together act as high sensitivity sensors in the chain length range from C8-CoA to C20-CoA. The Phe-49_Cys49 badan derivative of bovine ACBP which has the badan group exposed to the environment did not respond to addition of any of the ligands showing that the flourescent group preferably is located in the binding site in order to respond to ligand binding. However, it is envisaged that the signal moiety may be located in any position, where a change in the hydrophobicity of the environment takes place upon binding of the CoA ester. The lack of or very low response of FACI53 and FACI24 respectively to CoA binding makes these sensors preferred high sensitivity sensor for any acyl-CoA producing enzyme including acyl-CoA synthetases. This makes FACI24 a very potent sensor in determination of total free fatty (FFA) acid concentration in any biological fluid following the conversion of these to acyl-CoA esters.

Pre-Treatment of Hydrophobic Analytes Other Than Hydrophobic CoA Esters

The method, construct and kit according to the invention may be used for measuring the concentration of a number of different hydrophobic analytes. The analytes all have in common that it is possible to convert them via known and simple methods to hyrdophobic CoA esters, which are the keypoint linking these analytes together.

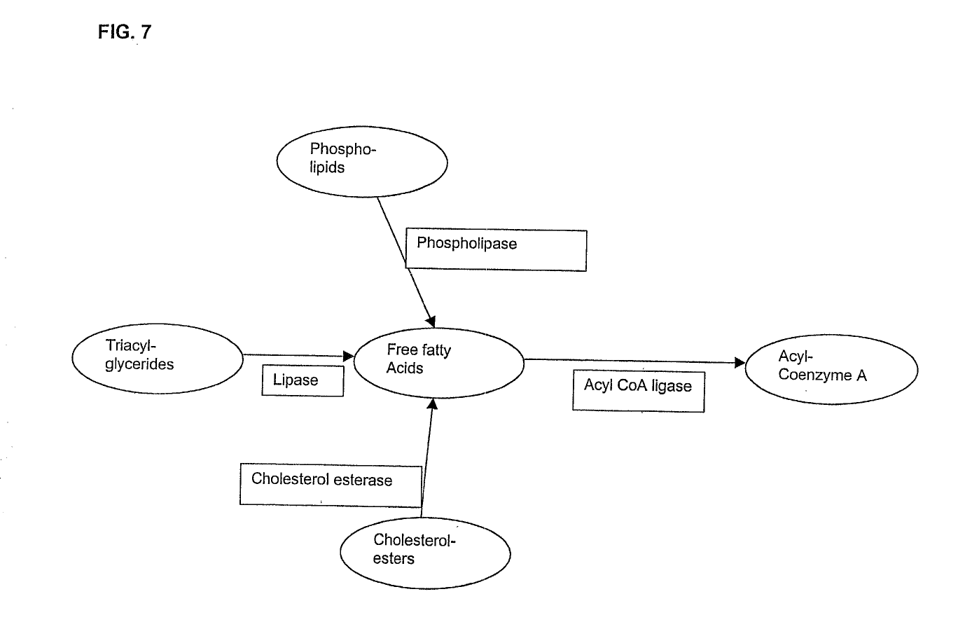

Reference is made to FIG. 7, in which the various groups of possible analytes are described together with suitable steps to perform before measurement of the amount of hydrophobic CoA ester. In the figure, ellipsoids contain the name of the different groups of hydrophobic analytes, triacylglycerides, phospholipids, cholesterolesters, free fatty acids and acyl CoA esters. Arrows show the direction of the steps necessary for converting the analytes into hydrophobic acyl CoA esters. The conversion steps may be performed in different ways, but for illustrative purposes the name of a preferred enzyme capable of catalysing the conversion steps have been added in rectangles.

A key reaction in the analysis of all hydrophobic analytes is the conversion of free fatty acids to acyl CoA esters. Methods based on initial conversion of the FFA to acyl-CoA are well known in the art. However quantification of the synthesised acyl-CoA in all the reported methods rely on time and resource consuming enzyme linked assays.

In order to be able to measure the concentration and/or presence of free fatty acids and/or lipids and/or phospholipids, these compounds must first be converted into CoA esters. Therefore the assay may further comprise a step prior to binding of the CoA esters with the construct, wherein hydrophobic acids in the sample are converted to hydrophobic Coenzyme A esters.

This conversion may conveniently be performed using enzymes such as acyl Coenzyme A ligase.

In all known FFA assays based on conversion of FFA to CoA esters pyrophosphatase is added to the sample together with acyl-CoA ligase and free CoA in order to drive the reaction in the direction of formation of CoA esters. By linking the cleavage of pyrophosphate liberated from CoA upon esterification to the acid group of the hydrophobic acid, to the esterification reaction, the overall reaction is rendered endothermic and essentially all hydrophobic acid is converted to hydrophobic CoA esters. However, because of the high binding affinity of the product of the esterification reaction towards the construct according to the present invention, this binding alone suffices to drive the esterification reaction. In the presence of the probe, the addition of pyrophosphatase may thus be dispensed with. In all other known assays, which include esterification of hydrophobic organic acids with CoA, pyrophosphatase is required to drive the reaction.

If fatty acids comprised in lipids are to be measured an additional step may be included during which triacylglycerides in the sample are converted to glycerol and free fatty acids. This hydrolysis is followed by esterification through acyl-CoA ligase.

The hydrolysis preferably is catalysed by lipase but it may also comprise acid or basic ester hydrolysis. Hydrolysis catalysed by lipase is by far the most gentle method and due to the specificity of the reaction the risk of uncontrolled and undesirable side reactions can be minimised. Thus lipase and the necessary reagents and co-factors may be added to the sample together with the components for the CoA binding assay.

In the case of phospholipids, the method preferably further comprises a prior step wherein phospholipids in the sample are converted to glycerol and free fatty acids. This is preferably performed using phospholipase A1 and/or phospholipase A2 but may likewise comprise acid or basic ester hydrolysis.

The inventors also envisage that the method may be used for estimation of the concentration of cholesterol esters in a sample such as a blood sample. The amount and type of cholesterol esters in blood is indicative of several diseases such as aterosclerosis and genetic defects such as familiar hypercholesterolemia. After cleaving cholesterolesters with an enzyme specific for cholesterolesters, the liberated free fatty acids may be combined with CoASH to form a CoA species that may be measured according to the present method.

Through combination of the various different pre-treatment steps, information concerning the type and amount of free fatty acids, CoA fatty acid esters, fatty acids making part of triacylglycerides and fatty acids constituting part of phospholipids in one and the same sample may be obtained.

Such combined assay may first comprise measurement of the amount of CoA esters in the sample using the construct according to the invention. By addition of acyl-CoA ligase the amount of free fatty acids may then be measured. Then the amount of triacylglyceride fatty acid may be measured by addition of lipase, and finally the amount of phospholipid fatty acids may be measured through addition of phospholipase A1 and/or phospholipase A2.

Other Types of Pre-Treatment

When assaying biological fluids for free hydrophobic acids or lipids, these have to be separated from present cells and cellular components and from proteins, which may interfere with the assay.

According to one aspect of the invention, this is carried out by mixing the sample with a water-miscible organic solvent after removal of cells and cell debris from the sample. Through addition of the water-miscible organic solvent, the proteins precipitate and the free hydrophobic acids and lipids, phospholipids, cholesterol esters and the like will remain in solution in the solvent, water mixture.

A small sample of the protein- and cell-free extract can be transferred to an assay mixture in a multi-well dish or the like. The dilution performed at this step is enough to dilute the water-miscible organic solvent to an extent where it does not interfere with the binding assay. Of course this extraction method can be used with other types of free fatty acid assays such as chromatographic assays, HPLC, gas chromatography and binding to a fluorescently modified fatty acid binding protein.

Accordingly there is provided a method for determining the amount of free hydrophobic carboxylic acid(s) and/or lipid constituent(s) in a sample comprising

i. optionally fractionating the sample to obtain a substantially cell-free sample,

ii. mixing the substantially cell-free sample with an amount of water-miscible organic solvent to precipitate proteins and obtain a solution of free fatty acids,

iii. subjecting a sample of the supernatant to a quantitative analysis determining the amount of free fatty acids in the sample.

The method may be performed on any kind of sample, including solid samples that are to be homogenised to extract hydrophobic acids and lipids. Preferably the method is performed on blood, urine, milk, tears, faeces, sperm, cerebrospinal fluid, nasal secrete, food, feed and mixtures, dilutions, or extracts thereof.

According to an especially preferred embodiment the method is performed on a blood sample and the substantially cell-free sample is serum. Hydrophobic acids are often assayed in blood samples for diagnostic purposes and therefore there is a need for simple procedures for analysing such samples.

Examples of water-miscible organic solvents that may be used include but are not limited to the group consisting of acetone, acetonitrile, dioxane, dimethyl sulfoxid, dimethyl formamide. These solvents are all miscible with water and serve the dual purpose of denaturing proteins and dissolving hydrophobic acids and lipids.

Preferably the solvent used is an alcohol, more preferably a low molecular weight alcohol. Such solvents are available at low prices and in the necessary purity and are do not interfere with the majority of quantitative analyses.

In the present context a low molecular weight alcohol may be defined as an alcohol having 1, 2 or 3 carbon atoms, such as ethanol, methanol, 1-propanol, 2-propanol, and cyclopropanol.

Preferably the low molecular weight alcohol is selected from the group consisting of ethanol and 1-propanol. These are the most preferred compounds due to the low cost, relatively low toxicity to human beings and relatively low vapour pressure compared to e.g. methanol.

More preferably the low molecular weight alcohol is ethanol. A suitable source of ethanol is abs. ethanol or ethanol having a concentration of 96% (v/v) ethanol.

After extraction the free hydrophobic acids or lipids may preferably be assayed according to the methods disclosed in the present invention. However the extraction method may also be used together with quantitative analyses such as gas-chromatography, HPLC, or binding to a fluorescently modified fatty acid binding protein.

Applications

The sensitivity and the simplicity of the constructs according to the invention make them useful in a variety of applications. At present no other methods exist for determining free acyl-CoA concentration. The probes are able to monitor the rate of C8- to C20-acyl-CoA production by any such acyl-CoA producing reaction. The probes are also in combination with acyl-CoA synthetase able to monitor the release of fatty acids (C8 to C20) from fatty acid producing reactions. The advantage of the present probes for determining FFA in combination with acyl-CoA synthetase over the ADIFAB probe produced by Molecular Probes is that the FACI24 and FACI53 are specific for long (>C12) and the medium chain acyl-CoA (C8 to C12) respectively. Furthermore the method does not require knowledge about the concentration of fatty acid binding proteins such as albumin in the reaction mixture.

As illustrated above it will not be difficult for a skilled protein chemist following procedures presented herein to construct new CoA or acyl-CoA probes using the above and other variants of ACBP. It only requires introduction of a environmentally sensitive signalling group in a position in the binding site which undergoes environmentally changes upon ligand binding. In the present study the amino acid residues selected to be mutated and derivatised have been shown to interact directly with the acyl-chain of the bound ligand (Kragelund,et. al., 1999; Biochim Biophys Acta. 1441, 150-161). These residues are exposed to the solvent in the unbound protein. The down shift in emission spectra therefore represents a hydrophobic shift in the local environment upon ligand binding. The ligand binding site is an open bowl like cavity from which water is displaced and the hydrophobic binding pocket for the acyl-chain is formed by the protein and the CoA head group together upon ligand binding (Faergeman, et. al.,1996; Biochemistry. 35:14118-14126; Kragelund, et. al., 1193; J Mol Biol. 230,1260-1277). A more sensitive probe would be one where the environment of the flourescent group is undergoing more dramatic changes upon ligand binding.

The use of the probes presented herein is the only existing way to measure free acyl-CoA concentrations of the physiological important, highly amphiphatic, medium and long chain acyl-CoA esters. Long-chain acyl-CoA esters partition in to membranes, stick to proteins and test tube cell walls. All previous published methods measure total acyl-CoA concentration including the very small fraction of free acyl-CoA, the biological active fraction, which can only be measured with the probes invented herein. From the literature it is clear that knowledge of the free acyl-CoA concentration in vivo and in vitro conditions is the key to understand the function of these very important molecules in regulation of key cell functions including gene expression (Faergeman and Knudsen, 1997; Biochem J. 323,1-12). The advantage with the present probes are their high degree of specificity for hydrophobic-CoA esters only. The CoA head group determines the binding specificity of ACBP by interacting with specific amino acid residues in the binding site and contribute with 50% of the binding energy (Færgeman, et.al. 1996; Biochemistry, 35, 14118-26). ACBP does not bind fatty acids, nucleotide, prostaglandins and a number of other compound tested (Rosendal, et. al., 1993, Biochem J. 290,321-326). The high specificity makes the probes very suitable for both in vitro and in vivo studies. The present work demonstrate the values of the FACI probes for in vitro determination of free acyl-CoA concentration. It is also envisaged and within the scope of the present invention to use the probes for in vivo studies.

The exemplary method, fluorescence ACBP, will also have wide applicability in studies of intracellular acyl-CoA transport, the role of acyl-CoA esters in fatty acid induced diseases and in enzymatic assays measuring total fatty acid concentration and the rate of fatty acid release from lipases, cells and lipid degradation in feed and food preparations.

The Sample

The method and the assay according to the invention may be used on any sample type. The ease of the method combined with the high specificity and the absence of cross reactivity with other components of the sample, make the method especially suited for direct analysis of complex samples without any preceding purification step. Accordingly the method may advantageously be performed on samples selected from the group consisting of blood, urine, milk, tears, faeces, sperm, cerebrospinal fluid, nasal secrete, food, feed and mixtures, dilutions, or extracts thereof. More preferably the sample is selected from group consisting of blood, urine, milk, food and feed and mixtures, dilutions, or extracts thereof.

According to an especially preferred embodiment, the measurement of hydrophobic CoA esters is performed directly on blood or serum samples and dilutions or extracts thereof. More preferably this method comprises the determination of total lipids and/or free fatty acids in the blood or serum.

It is also envisages that the method according to the invention may be useful for measuring the level of hydrophobic CoA esters or the measurement of lipids and/or fatty acids in milk and dilutions or extracts thereof.

Due to the methods simplicity, it is also envisaged that the method according to the invention will be used for one step measurement of lipids and/or fatty acids in samples comprising food and dilutions or extracts thereof as well as in samples comprising feed and dilutions or extracts thereof.

The sample may also comprises urine and dilutions or extracts thereof. The presence of free fatty acids in the urine may be indicative of various diseases, among them the deficiency known as “mitochondrial medium chain acyl CoA dehydrogynase deficiency”, which results in the presence of dicarboxylic acids of 8 of 12 carbon atoms in the urine.

The inventors have also determined that the constructs according to the invention are especially useful for determining the insulin sensitivity and the rate of lipolysis by adipocytes. Thereby, the constructs and the method may be used for early diagnosis of diabetes. Through an early diagnosis of diabetes, diet and diabetic treatment may be initiated early, and the occurrence of symptoms of diabetes such as blindness, macrovascular diseases such as generalised arteriosclerosis, hypertension, myocardial infarction, stroke, or microvascular diseases such as retinopathy or nephropathy, or neuropathy may be avoided or delayed. Avoidance and/or postponement of these symptoms have profound implications for the individuals suffering of diabetes and also results in enormous savings on public healthcare.

Assay Kits

The construct and the method according to the invention may advantageously be combined in a kit for determination of the concentration of hydrophobic CoA esters in a sample. According to one aspect of the assay kit, the reagents may be loaded into a multiwell dish to which the sample is added and the assay performed. The detection may subsequently be performed in a multi-well reader.

In its simplest form, the assay kit is adapted for determination of the concentration of hydrophobic Co-A esters. In order to be useful for the determination of free fatty acids the kit may further comprise an acyl-Coenzyme A synthetase, coenzyme A, adenosinetriphosphate, Mg⁺⁺, an antioxidant, and buffer. If the thioesterification is carried out in the presence of a construct according to the invention, there may be no need for pyrophosphatase to drive the thioesterification reaction. If the thioesterification is carried out spatially separate from the construct according to the invention, pyrophosphatase may advantageously be added to drive the thioesterification.

The kit according to the invention may also be adapted for determination of total lipids in which case it preferably comprises a lipase, and buffer to hydrolyse the triacylglycerides. For the determination of phospholipids the kit may comprise a phospholipase such as phospholipase A1 and/or A2, and buffer.

All the compounds used for the kits according to the invention may advantageously be freeze dried.

In some cases, especially those where the construct is not located in the sample compartment, it may be advantageous to add albumin to the kit. The presence of albumin ensures that free fatty acids and/or hydrophobic Co-A esters do not bind to the surfaces of the sample compartment. The albumin furthermore may be used for carrying the hydrophobic Co-A esters through a wick to immobilised constructs according to the invention.

The above described kits may either comprise a kit, wherein essentially all reagents (including the constructs according to the invention) are added to the sample compartment before addition of the sample.

Alternatively the assay kit may comprise at least one construct according to the invention, being immobilised on a solid support such as an extended solid phase. Such kits are known in the art under several names such as “lateral flow devices”, or dip sticks. Illustrative and not limiting examples of suitable lateral flow devices that may be used in accordance with the present invention include those described in U.S. Pat. No. 5,686,315 (PRONOVOST), U.S. Pat. No. 4,943,522 (EISINGER et al), U.S. Pat. No. 4,703,017 (BECTON DICKINSON) U.S. Pat. No. 4,855,240 (BECTON DICKINSON), U.S. Pat. No. 5,798,273 (BECTON DICKINSON). The extended solid phase is preferably of a type that allows a liquid sample comprising an analyte to diffuse through it without substantially binding the analytes or lowering the rate of movement of the analyte through the porous solid phase.

The extended solid phase may be in the shape of a dipstick, which may be dipped into a liquid sample, or it may have on it a sample compartment for applying a volume of sample, preferably a pre-determined amount of liquid sample. The kit may thus comprise a sample compartment and in another location a read out area in which constructs according to the invention are immobilised to the porous support phase and provide a signal when bound to hydrophobic CoA esters. The kit may be comprised in a housing with a hole for application of the sample into the sample compartment and a window for the read out area. After application of sample to the sample compartment, liquid sample moves through the porous solid support past the read out area to the end of the kit. The porous material may be any material to which the constructs can be linked, and which does not interfere with the assay such as through binding of free fatty acids, lipids or hydrophobic CoA esters. One suitable material may be nylon or nitrocellulose paper.

When the construct is immobilised, the sample may be added to the sample compartment, where it is optionally subjected to lipase and/or phospholipase and/or acyl-CoA ligase. The sample compartment advantageously also comprises albumin. After pre-treatment of the sample has been performed, the sample may be allowed to move via a wick to the immobilised construct. When pre-treatment is carried out in a sample compartment connected to the porous solid support, the kit preferably comprises means to seal the sample compartment from the porous solid support in order to avoid movement of sample through the porous support before the pre-treatment steps are concluded.

However, the pre-treatment may also be performed in another location such as in a test tube in order to avoid movement of liquid sample through the porous solid support before the pre-treatment steps are concluded.

As the liquid front reaches the immobilised constructs, the hydrophobic-CoA esters will be bound to the immobilised construct and the detection may be performed. Advantageously, the hydrophobicl-CoA esters are bound to albumin as they diffuse through the wick to the imrnobilised construct. As the affinity of the constructs according to the invention to is much higher than the affinity of albumin, albumin will deliver the CoA esters to the constructs.

The kits wherein the construct is immobilised may comprise constructs which are immobilised in at least two different places, such as at least 3, for example at least 4 such as at least 5 different spaces. In the case, where the constructs are identical, this embodiment is useful for rapid, one-step determination of the concentration of hydrophobic CoA esters or free fatty acids or lipids in a sample. A pre-determined amount of construct according to the invention, capable of binding a pre-determined amount of hydrophobic CoA esters is immobilised in two, three, four, five or more spaces on a stick. A predetermined amount of sample is added to the end of the stick after appropriate pre-treatment. As capillary forces move the liquid sample past the immobilised constructs, a pre-determined amount of hydrophobic CoA esters is bound to the immobilised constructs causing a change in the signal emitted from the constructs. As the liquid front has moved past all locations of immobilised construct the signals are detected. The larger the amount of hydrophobic CoA esters in the sample the more of the locations of construct will emit a signal indicative of bound CoA esters.

The kit according to the invention, may also comprise more than one construct such as a second hydrophobic-Coenzyme A ester binding construct, or at least a third construct, such as at least a third and a fourth construct, for example at least a third, a fourth and a fifth construct. It is to be understood that these constructs have a high binding affinity for different species of CoA ester or for a different group of CoA esters. By allowing a liquid sample to pass the immobilised constructs, different CoA esters will bind to different constructs. Upon detection, the presence of several species or groups of species may be detected. Through measurement of the intensity of the signals, the relative amount of different species and/or groups of species may be determined. Preferably each construct has a K_{D} with respect to at least one species or a group of species of hydrophobic Coenzyme A esters, which is substantially lower than the K_{D} of the other construct(s) with respect to this species or group of species.

According to a preferred embodiment of the invention, substantially lower may be 10 times lower, more preferably 100 times lower.

As a non-limiting example a kit according to the invention may comprise the first construct being a fluorescence acyl-CoA sensor 1 (FACI 24) and a second construct being a fluorescence acyl-CoA sensor 2 (FACI 53).

Coding Sequences/Expression Vectors

The heterologous peptide comprised in the construct according to the invention may conveniently be manufactured using recombinant techniques. The invention therefore also features a nucleotide sequence encoding this heterologous peptide. Recombinant techniques for preparing nucleotide sequences are well known to the skilled practitioner.

The nucleotide sequence may be inserted into an expression vector, which is used for transformation of a cell. Eventually the cell comprises the nucleotide sequence encoding the peptide part of the construct under the control of a suitable promoter. The construct may be manufactured by the cell, harvested and optionally purified further prior to addition of the signal moiety.

Alternatively the peptide may be manufactured using well known chemical synthesis methods.

The invention is now illustrated with a number of examples, which are in no way to be interpreted as limiting to the scope of the invention, which is determined by the claims.

### EXAMPLE 1

### Site-Directed Mutagenesis Using the QuikChange (Stratgene)

Template (50 ng of Bov-ACBP in pET3a) was incubated in Pfu Turbo reaction buffer (20 mM Tris-HCl, pH 8.8, 10 mM KCl, 10 mM (NH₄)₂SO₄, 2 mM MgSO₄ 0.1 % Triton X-100, 0.1 mg/ml BSA) supplemented with 0.25 mM dNTP, 125 ng of each mutagenic primer and 2.5 U Pfu Turbo polymerase in final volume 50 ml. The reaction was cycled using the following parameters: 95° C./30 s, 55° C./2 min, 68° C./10 min for 16 cycles. Subsequently, the reaction was placed at 37° C. and 10 U of Dpnl was added to remove the parental DNA and incubated for at least 1 hour. Finally the DNA was transformed into competent DH5a cells. Ampicillin resistant transformants were selected and plasmids were purified using the plasmid kit from Qiagen. Plasmids were sequenced using the CEQ DTCS kit (Beckman) as described by the manufacturer. Plasmids containing the desired mutation were transformed into BL21(DE3)pLysS and protein was induced and purified as described previously.

The recombinant Met24_Cys24-, Phe49_Cys49- and Ala53_Cys53-bovine ACBP were fluorescently labelled with 6-bromoacetyl-2-dimethylaminonaphthalene (Badan, Molecular Probes), Badan was used because of its sensitivity to polarity of its environments and which was expected to make it particularly sensitive to interaction of hydrophobic-CoA esters with ACBP and because Badan is capable of covalent modification of protein amino acid residues. To carry out the reaction, 1.2 mole excess badan over ACBP was added over 10 min by continuous infusion from a 20 mM stock solution of Badan in dimethylformamide, to a 1 mg/ml solution of Met24_Cys24-, Phe49_Cys4-, or Ala53_Cys53-bovine ACBP in 50 mM tris/HCL pH 7.2. Incubation was continued for 15 min and the reaction was stopped by addition of excess DTT. Unreacted badan and badan side reaction products were removed by passing the reaction product over a 1 ml Lipidex-1000 column. The resulting derivatised protein was shown to have a stochiometry of 1 badan per mole protein by electrospray mass spectrometry. The localisation of the badan derivatised amino acid was confirmed by tryptic digestion and separation of the tryptic peptides by reverse phase HPLC using water/acetonitrile/TFA solvent system followed by mass determination and sequencing of the flourescently labelled peptide.

### EXAMPLE 2

### Dissociation Constant of the Construct/Ligand Complex

Quantitative determination of binding affinities (K_{D}) were performed by isothermal titration micro calorimetry as previously described (Faergeman et. al., 1996; Biochemistry 35, 14118-14126). Qualitative evaluation of relative binding affinities were determined by isoelectrical focusing using the Pharmacia Fast Gel system according to the prescriptions given by the manufacture. Fluorescence emission changes induced by acyl-CoA binding to badan modified protein were determined as follows: 5 μl portions of acyl-CoAs were added from stock solutions disolved in binding buffer, (10 mM Hepes, 150 mM NaCL, 1 mM NaHPO₄, pH 7.4) containing 3.4 μM Badan derivatized protein to a 1 ml 3.4 μM solution of the Badan derivatized ACBP in the same buffer. The fluorescence emission was measured on a SPEX FLOUROLOG (Industries Inc, Edison N.J., USA) with excitation at 400 nm and emission scan from 400 nm to 550 nm. The concentration of acyl-CoA in the aqueous phase was determined from the fluorescence emission sensitivities at 495 and 470 nm repectively essentially by the method described by (Grynkiewickz et. al.,1985, J. Biol. Chem. 260, 3440-3450) according to which:

[acyl-CoA]_{free}=K_{D}((F−Fₘᵢₙ)/(Fₘₐₓ−F)

Where F is the measured fluorescence in the solution and Fₘᵢₙ the fluorescence in the absence of ligand and Fₘₐₓ the fluorescence in the presence of saturating ligand concentration.

The exclusive binding of hydrophobic-CoA esters by ACBP is determined by specific recognition of the CoA head group (Kragelund, et. al.,1993; J Mol Biol, 230(4):1260-1277) CoA itself is bound with low affinity (K_{D}=2 μM) with increasing acyl-chain length the affinity increases K_{D}˜1-2 nM) up to 22 carbons after which the binding affinities drop dramatically (Faergeman et. al., 1996; Biochemistry 35, 14118-14126; Rosendal, et. al.,1993; Biochem J. 290, 321-326; Robinson, C. V., 1996. J. Am. Chem. Soc., 118, 8646-8653). The mutated amino acid residues were chosen as residues which have been shown to interact with the acyl-chain of the bound acyl-CoA in the ACBP/acyl-CoA complex (Kragelund, et. al.,1993; J Mol Biol, 230(4):1260-1277 ). The primary structure of ACBP is highly conserved throughout eukaryote from S. pompe to man and the basic structure and binding properties is expected to be very similar in ACBP from all species (Kragelund, et. al., 1999, Biochim Biophys Acta. 1441, 150-61). The obtained reltsults with the Badan derivatised bovine ACBPs are therefore expected to be representative for ACBP from all species. We are presently making the Met24_Cys24-badan analog of Yeast and rat ACBP to confirm this.

### EXAMPLE 3

### One Step Assay of FFA

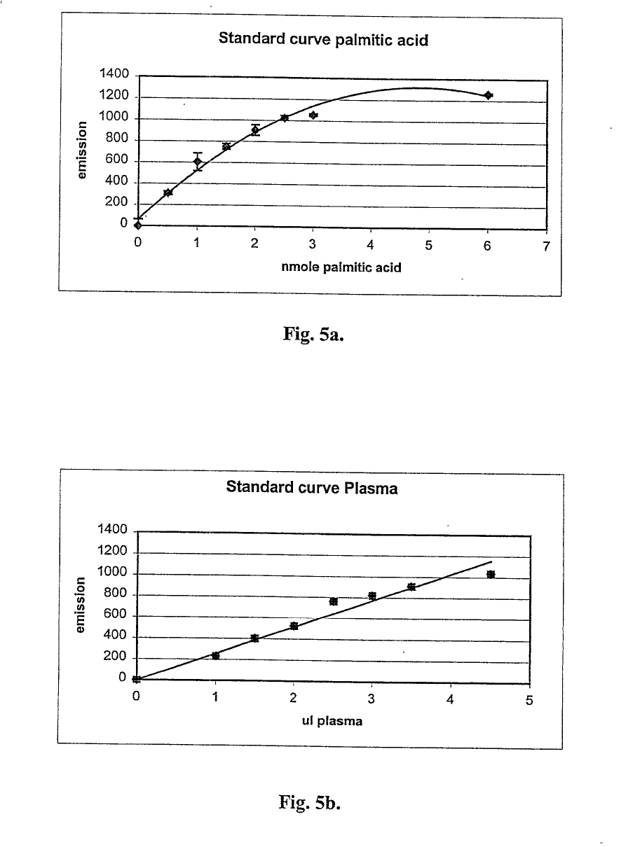

To demonstrate the ability of FACI24 to act as a sensor for determining the level of total free non-esterified fatty acids in biological fluids FACI24 (4 μM) was incubated in a reaction mixture containing: 100 mM Tris/HCL pH 7.4, 1 mm DTT, 2 mM EDTA, 4 mM Mg(CL)₂, 4 mM ATP, 60 μM CoA, 0.03 units/ml Acyl-CoA synthetase and 0.06 units/ml Pyrophosphatase at 37° C. for 30 min. The reaction was started by addition of human serum or free fatty acid standard bound to equimolar amounts of bovine serum albumin. The results in FIGS. 5A and B show that the present invention makes it possible to determine the formed acyl-CoA in a one step reaction simultaneously with the formation of the acyl-CoA esters by the acyl-CoA synthetase direct in the reaction mixture. The use of FACI24 to determine the formed acyl-CoA esters increase the sensitivity of present methods and make it possible to determine FFA in less than one micro liter of serum (FIG. 5). A total fatty acid method based on the FACI24 sensor will be of great value, it will simplify present assays and make it possible to measure total fatty acids in body fluid from even very small species and infant.

The standard curve in FIG. 5A was prepared using the following mix of reagents:

| | | |
|---|---|---|
| M24C-BADAN | 3 | μM |
| CoA | 60 | μM |
| MgCl₂ | 4 | mM |
| EDTA | 2 | mM |
| AcylCoA synthetase | 0.03 | units/mL |
| Pyrophosphatase | 0.06 | units/ML |
| Tris/HCl, pH 7.4 | 100 | mM |

1 mL of the reaction mix was added to different amounts of 50 μM palmitic acid (dissolved in 100 mM Tris/HCl, 50 μM bovine serum albumin) to a final concentration of 0.5, 1.0, 1.5, 2.0, 2.5, 3.0, and 6.0 μM. The mixture was incubated for 30 min at 37° C. and then the sample was excitated at 400 nm and the emission was measured at 470 nm.

The curve in FIG. 5B was prepared using the following mix of reagents:

| | | |
|---|---|---|
| M24C-BADAN | 4 | μM |
| CoA | 60 | μM |
| MgCl₂ | 4 | mM |
| EDTA | 2 | mM |
| AcylCoA synthetase | 0.03 | units/mL |
| Pyrophosphatase | 0.06 | units/ML |
| Tris/HCl, pH 7.4 | 100 | mM |

1 mL of the reaction mix was added to different amounts of plasma 0, 1, 1.5, 2.0, 2.5, 3.0, 3.5, 4.0, and 4.5 μL. The mixture was incubated for 30 min at 37° C. and then the sample was excitated at 400 nm and the emission was measured at 470 nm.

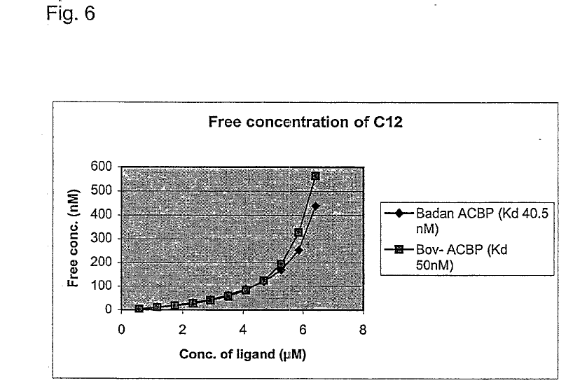

In order to test the ability of FACI24 to function as a sensor of free unbound acyl-CoA native bovine ACBP (5.58 μM) was titrated with dodecanoyl-CoA (C12-CoA) in the presence of FACI24 (0.58 μM) and the free C12-CoA concentrations were calculated from fluorescence emission changed of FACI24 induced by C12-CoA titration. The K_{D} for C12-CoA binding to M24C-badan ACBP, used in the calculation of free acyl-CoA concentration from fluorescence measurements, was determined to 40 nM using isotermal titration micro calorimetry. This concentration was compared with concentrations calculated from the predetermined K_{D} for C12-CoA binding to native bovine ACBP (Færgeman et al. Biochemistry. 1996 Nov 12;35(45):141 18-26. The results in FIG. 6 demonstrate that there is a good agreement between the calculated and the measured free acyl-CoA concentration measured using FACI24. This demonstrates that FACI24 functions as a sensor for measuring free acyl-CoA concentration.

The data presented herein demonstrate that FACI24 and FACI53 are highly specific and extremely sensitive probes for free non-esterified fatty after conversion to acyl-CoA esters and free C8- to C20-acyl-CoA esters in aqueous solution in the low nM range.

### EXAMPLE 4

### Site Directed Mutagenesis

Briefly, adjacent 5′-phosphorylated oligonucleotides were designed on opposite DNA strands with the mutation encoded at the 5′ end of the upstream primer. PCR mutagenesis was performed by using the bovine ACBP open reading frame in pET3a as template (10-50 ng), in a mixture consisting of 1.25 U pfu turbo polymerase (Stratagene), 50 pmol of each oligonucleotide, 200 μM of each dNTP, in pfu reaction buffer (20 mM Tris-HCl, pH 8.8, 10 mM KCl, 10 mM (NH₄)₂SO₄, 2 mM MgSO₄, 0.1% Triton X-100, 100 μg/ml bovine serum albumin). The primers used for amplification of individual mutants are shown in Table 1. Prior to amplification the template was denatured at 94° C./5 min, followed by 16 cycles of 94° C./1 min, 55° C./1 min, 72° C./13 min. Controls did not receive any polymerase. Subsequently, the amplicon was phenol-chloroform extracted and precipitated with ethanol. DNA was re-suspended in T4 ligase buffer (20 μl) (New England Biolabs) containing T4 ligase (10 U) and incubated 18 hours at 16° C. The samples were diluted to 50 μl in Dpnl digestion buffer and incubated with Dpnl(60 U) for 2 hours at 37° C. DNA (2-5 μl) was then transformed into CaCl₂ competent DH5α cells. Plasmid DNA was isolated by standard methods and plasmids carrying the correct change were identified by restriction analysis and DNA sequencing.

TABLE 1
Primers used for site directed
mutagenesis of bovine ACBP

| Mutation | Sequence |
|---|---|
| M24C | |
| Upsteam | 5′-TGCTTGTTCATCTACTCTCACTACAAG |
| Downstream | 5′-TTCTTCGTCGGCCGGCTTGGTCTTC |
| M46C | |
| Upsteam | 5′-TGCTTGGACTTCAAGGGTAAGGCTAAG |
| Downstream | 5′-CCCGGGTCTTTCGGTGTTGATGTC |
| A53C | |
| Upsteam | 5′-TGCAAGTGGGACGCTTGGAACGAATTG |
| Downstream | 5′-CTTACCCTTGAAGTCCAACATCCC |

For large scale production of recombinant protein the bacteria were grown in a 4 l fermentor. The cells were harvested by centrifugation and frozen at −80° C. Cells were thawed, resuspended in 0.9 % NaCl, 1 M acetic acid, sonicated and cleared by centrifugation for 20 min at 10000× g at 4° C. pH was adjusted to 7.0 with 1 M NaOH and precipitated proteins were removed by centrifugation as described above. The cleared supernatant (approximately ˜140-160 ml) was divided in two and was loaded on a Sephadex-G50 column (5 cm×80 cm, Amersham Pharmacia Biotech, Copenhagen, Denmark), equilibrated and run with 60 ml/h in 10 mM Tris-HCl pH 7.2. The eluate was collected in 12 ml factions. The fractions containing the ACBP peak were pooled, made 5% with freshly prepared TCA and centrifuged as described above. The protein pellet was resuspended and washed with 10 mM TCA and centrifuged again. The residual TCA was carefully removed and the protein pellet was dissolved in 30 mM Tris-base, 100 mM DTT and the pH adjusted to 7.2 with 30 mM Tris-base. The solution was cleared by centrifugation and stored at −80° C. Immediately before badan labeling the frozen supernatant was thawed and loaded on a Q-sepharose HP column (1.5 cm×12 cm, Amersham Pharmacia Biotech, Copenhagen, Denmark) equilibrated with 10 mM Tris-HCl pH 7.2 and bound proteins were eluted with a linear gradient from 0 to 400 mM NaCl with a flow of 3 ml×min⁻¹. The fractions containing ACBP were pooled, adjusted to 100 mM with Tris-HCl pH 7.2 and used directly for synthesis of badan derivatized ACBP.

Protein Expression and Modification

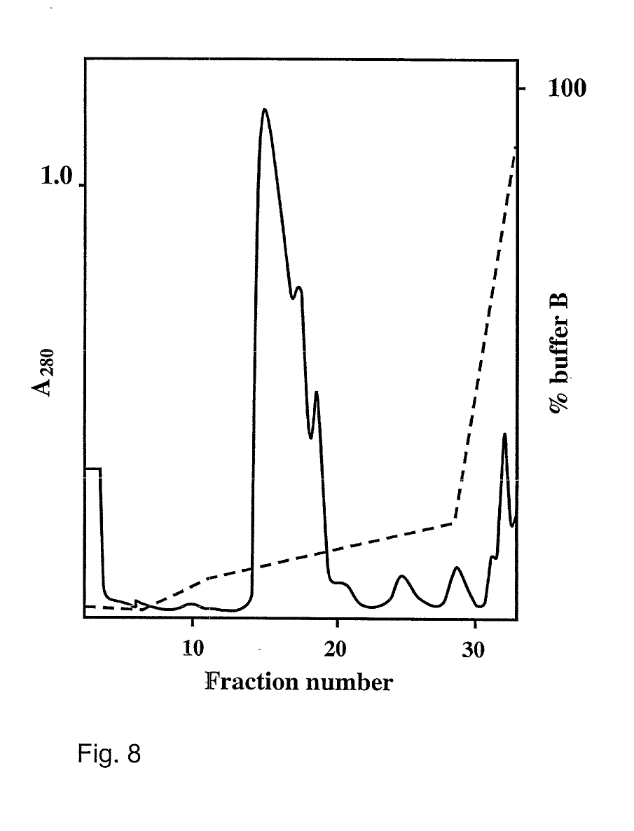

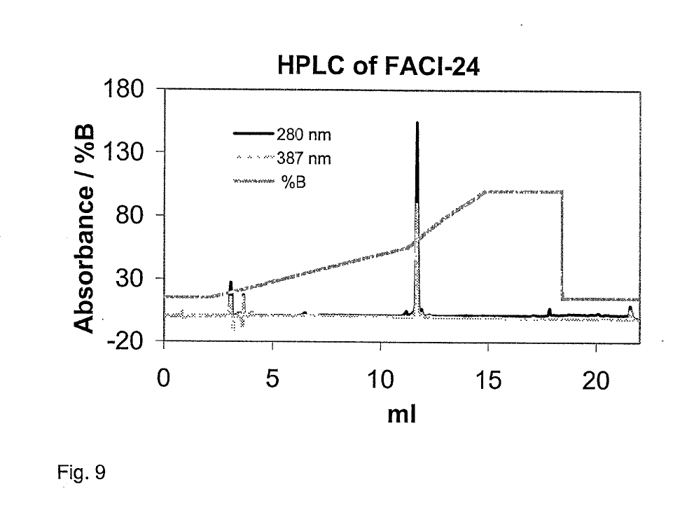

Expression of the cysteine modified bovine ACBP in E. coli DH5α with the gene inserted in the pKK233-3 expression vector as previously described for native ACBP was unsuccessful. The bacteria did not grow or expressed only low levels of any of the cysteine modified ACBP proteins indicating that they were toxic to DH5α. Expression of the cysteine containing proteins from the pET3a vector in the E. coli strain BL21(DE3)pLysS resulted in a high yield of recombinant protein. However, following purification we found that the introduced cysteine was partly modified, a phenomenon which was most pronounced with A53C-ACBP. Approximately 50% of the produced A53C-ACBP was esterified with CoA as shown by mass spectrometry (result not shown). M24C-ACBP contained two modified versions which could be separated from the unmodified form on Q-sepharose ion exchange chromatography (FIG. 8). The final unmodified product was shown to have the correct molecular weight as determined by mass spectrometry (10095 Da, result not shown). Non mutated recombinant bovine ACBP did not react with badan under the reaction conditions used (result not shown), however modification of the cysteine in M24C-ACBP caused a second group to react with badan. To prevent modification of this second group 1.1 molar excess of badan over M24C-ACBP was infused in to the reaction mixture at a controlled rate. Using this procedure the M24C-ACBP was found to be completely modified to give one product only (FIG. 9). The small amount of double derivatized protein was insoluble and was completely absorbed on the desalting column. Mass spectrometry showed that the final product only contained one protein with a molecular mass of 10095, confirming that M24C-ACBP only had been modified with one badan. Trypsin digestion of M24C-24-badan- ACBP (FACI-24) resulted in two badan derivatized peaks with a molecular weight of 2155.71 Da and 1865.26 Da. The unmodified tryptic peptide Thr-17 to Lys-32 of M24C-ACBP has a predicted molecular weight of 1943.19 Da. The molecular weight of the two peaks corrected for the badan group (211.09) is 1944.62 and 1654.17 Da. The 2155.71 Da peptide therefore correspond to the Thr-18 to Lys-32 peptide, and the 1865.26 peptide is the same peptide which has lost the C-terminal Thr-31 and the Lys-32. The present data therefore confirm that FACI-24 is derivatized with one badan at position Cys-24. The A53C-ACBP variant eluted as two completely separate peaks from the ion exchange column. Mass spectrometry showed that the second peak was 767 mass units too large, corresponding to the molecular mass of CoA. As expected this extra mass could be removed by incubation with 100 mM DTT. This indicates that 30-40% of Cys-53 in A53C-ACBP was esterified with CoA in E. coli.

M46C-ACBP came out with the correct molecular weight. Their badan derivatives FACI-46 and FACI-53 was synthesized and characterized as described for FACI-24 above.

### EXAMPLE 5

### Badan Labelling

The recombinant M24C-, M46C-, and A53C-bovine ACBPs were labelled with 6-bromoacetyl-2-dimethylaminonaphthalene. Synthesis and all handling of badan derivatised proteins was carried out under dim light. To carry out the reaction, 1.1× molar excess of badan over ACBP was added over a period of 10 min by continuous infusion from a 20 mM stock solution of badan dissolved in dimethylformamide, to a 5 mg/ml solution of the individual proteins. The progress of the reaction was monitored by HPLC by injection of small aliquots on a Jupiter 5 μ, C18, 300A column equilibrated with 10% acetonitrile in water, 0.05% trifluoroacetic acid (TFA). Elution was carried out using a gradient from 10% to 90% acetonitrile in water, 0.05% TFA over a period of 15 min. Badan infusion was continued until all non-derivatized ACBP had disappered. The reaction was then stopped by addition of 1 mM DTT and the reaction mixture desalted into water on a Sephadex-G25 column (5×25 cm) and freeze dried. The localization of the badan derivatised amino acid was confirmed by tryptic digestion and separation of the tryptic peptides by reverse phase HPLC using water/acetonitrile/TFA solvent system, followed by mass determination and sequencing of the flourescently-labeled peptide. The resulting Fluorescent modified Acyl-CoA Indicator were named FACI-24, FACI-46, and FACI-53 respectively to signify the mutated and modified amino acid residue.

### EXAMPLE 6

### Equilibrium Binding Analysis

Development of a mathematical expression describing the relationship between acyl-CoA concentration and FACI-24 fluorescence was performed essentially as by Richieri et al 1992, J Biol Chem, 267:23495-23501. However, where ligand binding to ADIFAB induces both a fluorescence intensity increase around 505 nm and a fluorescence intensity decrease around 432 nm, ligand binding to FACI-24 only causes an increase in fluorescence around 460 nm. Hence, the expression for the free concentration of acyl-CoA looks like:

$$\begin{matrix}[ acyl - CoA ] = {K}_{d} F - \frac{{F}_{min}}{{F}_{max} - F} , and & ( 1 ) \\ [ acyl - {CoA}_{bound} ] = [ FACI ] \frac{F - {F}_{min}}{{F}_{max} - {F}_{min}} . & ( 2 )\end{matrix}$$

Derivatisation of an expression for the fluorescence value as a function of the acyl-coA concentration was done using the Scatchard equation as a starting point:

$$\frac{1}{[ acyl - {CoA}_{bound} ]} = \frac{{K}_{d} / [ FACI ]}{[ acyl - CoA ]} + \frac{1}{[ FACI ]} . \\ Since [ acyl - CoA ] = [ acyl - {CoA}_{total} ] - [ acyl - {CoA}_{bound} ] and recalling Equation 2 : \\ \frac{1}{[ acyl - {CoA}_{bound} ]} = \frac{{K}_{d} / [ FACI ]}{[ acyl - {CoA}_{total} ] - [ acyl - {CoA}_{bound} ] } + \frac{1}{[ FACI ]}$$

$$χ$$

$$\frac{1}{[ FACI ] F - \frac{{F}_{min}}{{F}_{max} - {F}_{min}}} = \frac{{K}_{d} / [ FACI ]}{[ acyl - {CoA}_{total} ] - [ FACI ] F - \frac{{F}_{min}}{{F}_{max} - {F}_{min}}} + \frac{1}{[ FACI ]}$$

$$this equation can be solved for F , yielding : \\ F = - \frac{\begin{matrix}( {F}_{max} - {F}_{min} ) \sqrt{{K}_{d}^{2} + ( 2 [ acyl - CoA ] + 2 [ FACI ] ) {K}_{d} + {[ acyl - CoA ]}^{2} - 2 [ FACI ] [ acyl - CoA ] + {[ FACI ]}^{2}} + \\ ( {F}_{min} - {F}_{max} ) {K}_{d} + ( - [ acyl - CoA ] - [ FACI ] ) {F}_{max} + ( [ acyl - CoA ] - [ FACI ] ) {F}_{min}\end{matrix}}{2 [ FACI ]}$$

which has been used to fit the titration data.

### EXAMPLE 6a

### Ligand Binding

The strategy behind the design of the mutants was based on the tertiary structure of the bovine ACBP/acyl-CoA complex. Both Met-24 and Ala-53 interact directly with the acyl-chain of the acyl-CoA ligand, interacting with the omega-methyl group of long acyl-chains. Positioning of an environmentally sensitive group in one of these positions would therefore be expected to create ligand sensitive probes. Met-46 which is located outside the hydrophobic binding pocket in the loop between helix 2 and 3 was chosen to serve as a negative control which should not be sensitive to ligand binding.

Fluorescence titration emission spectra from 415 nm to 550 nm (excitation at 400 nm) were performed for CoA, C4:0-, C8:0-, C12:0-, C16:0-, and C20:0-CoA with all four mutated and badan modified proteins. FACI-46 did not show any emission spectra changes with any of the ligands (results not shown) and was therefore not investigated further.

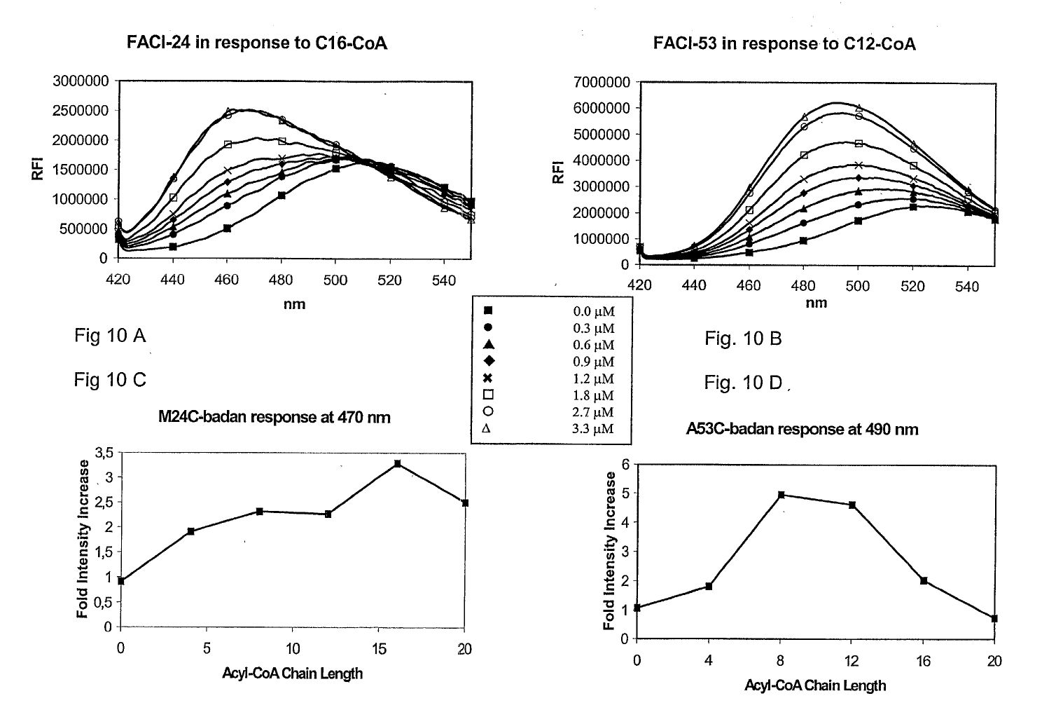

Titration of FACI-53 dissolved in binding buffer with the above mentioned acyl-CoA esters produced a smooth downward shift in fluorescence emission maximum from 525 nm when no ligand was present to 508, 503, 490, 492, 496 and 514 with increasing concentrations of CoA, C4:0-, C8:0-, C12:0-, C16:0- and C20:0-CoA respectively. The magnitude of emission and the relative emission at 495 nm was increased 1.1, 1.8, 2.9, 2.7, 1.3 and 0.8 fold and 1.5, 2.8, 4.8, 4.7, 2.2 and 0.8 fold respectively in the same order (FIG. 10) demonstrating that FACI-53 give the strongest signal for C8:0- to C12:0-acyl-chain.

The emission maximum of FACI-24 without ligand added was 510 nm and titration with CoA, C4:0-, C8:0-, C12:0-, C14:0-, C16:0- and C20:0-CoA caused 0, 16, 34, 42, 46 and 46 nm downshift in emission maximum, respectively (results not shown). C16:0-CoA induced a 5,5 fold increase in emission at 464 nm (FIG. 10).

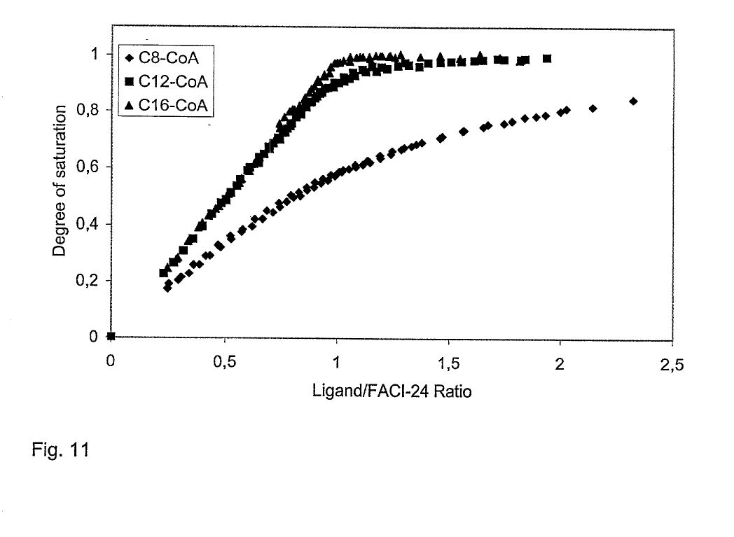

Titration of FACI-24 with increasing concentrations of C8:0-, C12:0- and C16:0-CoA resulted in a gradual increase in emission fluorescence at 460 nm (FIG. 11) providing sensitive measure for acyl-CoA binding. The curves represent the best fit analysis from two independent experiment calculated and normalized as described above. The Kd's for binding of the individual ligand to FACI-24 calculated from the binding curves are listed in table 2. The FACI-24 binding affinity increases dramatically when the acyl-chain length increases from C8 to C10 and C12, a smaller but significant drop in K_{d} is seen by increasing chain length from C12 to C14. C14 to C18 saturated acyl-CoA esters and C18:1-CoA binds with similar and very high affinities to FACI-24. In >0.1 M salt, FACI-24 binds acyl-CoAs with higher affinity and similar specificity as native unmodified bovine ACBP. The Kd for binding of C12-CoA and C16-CoA to native ACBP is 40 and 2.0 nM, respectively (Fulcery et al Biochemical, 1997, 325:423-428; Færgeman et al 1996, Biochemistry, 35:14118-14126). FACI-24 is highly specific for binding long-chain acyl-CoA esters. Free fatty acids do not bind and do not affect fluorescent emission at all at any wavelength (result not shown).

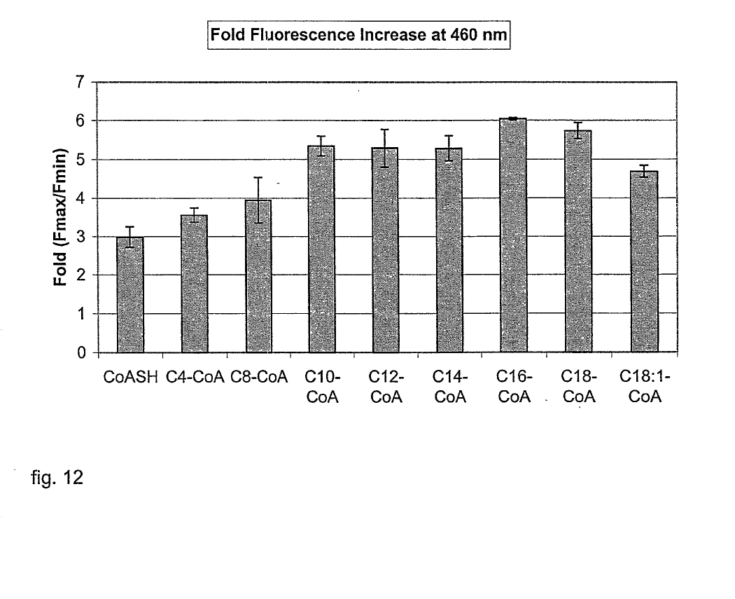

FACI-24 binds free CoA with a similar low affinity (K_{d}=2.5 μM as native bovine ACBP (K_{d} 2 μM). CoA induces a smaller increase (3 fold) in FACI-24 fluorescence emission at 460 nm than C14- to C18-CoA's which induces a 5 to 6 fold increase in fluorescense. The calculated relative emission changed induced by binding CoA to C18:0-CoA to FACI-24 at ligand/protein molecular ratios of 1:1, 1:1,5 and ∞ are shown in FIG. 12.

The shift in the fluorescence emission maximum from 525 nm to 460 upon ligand binding indicates that the badan group is shifted from a more hydrophillic environment to a more hydrophobic environment upon ligand binding. The fact that the interaction of C12- to C18-CoA esters results in very similar increases in the emission yield at 460 nm independent of acyl-chain length indicates that it is either the early part of the acyl-chain or the CoA head group which interacts with the badan group. This is surprising because Met-24 has been show to interact with carbon 12 to 16 of the ligand bound to native bovine ACBP. The explanation of this discrepancy will have to await determination of the tertiary structure of apo and holo FACI-24 which is in progress.

The very high fluorescent yield and binding affinity for acyl-CoAs with chain-length >C12 (K_{d}'s 1-2 nM) combined with the low binding affinity (K_{d} 2 μM) and low fluorescense yield with CoA, makes FACI-24 a potential sensor for quantification of mixtures of C12 to C20-CoA esters synthesized by acyl-CoA synthetase (ACS).

TABLE 2
Dissociation constants for binding of CoA and acyl-CoA esters to
FACI-24. FACI-24 (1.5 μM) was titrated with increasing CoA and
acyl-CoA concentration as shown in FIG. 11. The data was fitted
and Kd calculated as described in Examples 6 and 6a.

| Ligand | K_{d} (nM) ± S.E. |
|---|---|
| CoASH | 2448.79 ± 248.63 |
| C4:0-CoA | 1496.88 ± 222.65 |
| C8:0-CoA | 342.07 ± 31.90 |
| C10:0-CoA | 61.80 ± 2.49 |
| C12:0-CoA | 10.20 ± 0.81 |
| C14:0-CoA | 1.66 ± 0.26 |
| C16:0-CoA | 0.65 ± 0.26 |
| C18:0-CoA | 1.65 ± 0.83 |
| C18:1-CoA | 0.59 ± 0.19 |

### EXAMPLE 7

### Titration Data Analysis

Acyl-CoA Titration of FACI

Titration of FACI with acyl-CoAs was done using a SPEX FLOUROLOG (Industries Inc, Edison N.J., USA) with excitation at 387 nm and emission at 460 nm, with both excitation and emission slits set to 4.5 nm. FACI (0.5-4.5 uM, as indicated) was dissolved in 1.5 ml binding buffer (10 mM HEPES 150 mM NaCl, 5 mM KCl, 1 mM Na₂HPO₄, pH 7.4) and titrated with a 10 μM acyl-CoA dissolved in binding buffer, with or without FACI added, as indicated. Fluorescence emission values (counts per second) measured without FACI added to the ligand stock solution were corrected for titrand dilution.

Fluorescence values were represented as a function of the FACI-24 to acyl-CoA ratio, and an attempt to fit these data to the mathematical model was performed using DataFit® 7.1 software (Oakdale Engineering, Oakdale, Pa., USA; http://www.curvefitting.com). Up to four parameters were fitted in this way, these were B, representing the point of saturation on the X axis, H, representing the maximum fluorescence, i.e. the fluorescence at saturating concentration of acyl-CoA, L, the minimum fluorescence value, i.e. the fluorescence of FACI-24 alone in buffer, and K, the Kd value lor the acyl-CoA in question. Initial estimates for the fittable parameters were typically: B=1, H=maximum fluorescence value measured, L=minimum fluorescence value measured (at acyl-CoA-concentration=0), and K as judged from the raw data, typically from 0.0001 uM to 0.1 uM for acyl-CoA longer than C10:0-CoA.

For each acyl-CoA at least two titrations and fittings were performed. To consider two or more titrations of the same acyl-CoA together and obtain a Kd value using all these titrations, both axes of the raw data were standardised using the fitted values of B, H and L in this way:

X axis standard=(raw X axis values)/Bfitted

Y axis standard=[(raw Y axis values)−Lfitted]/[Hfitted−Lfitted]

In this way all Y axis values should come to lie between 0 and 1 and the X axis value should be 1 at the point of saturation (FACI-24:acyl-CoA 1:1). Such adjusted data were pooled and fitted using DataFit. Typically, both H and B fitted to values close to 1 and L fitted to values close to 0. K was typically fitted to values somewhere between the two values obtained for the raw data.

For short (≦C8-CoA) acyl-CoAs, titration curves were found to lack a significant point of saturation, causing the fitting procedure to often yield very high B values (2-3). This could be overcome by removing B from the fittable parameters list and instead setting it to a constant value of 1 and fitting again. This was typically done for both the raw and the standardised data.

### EXAMPLE 8

### Determination of Acyl CoA-Synthetase Activity

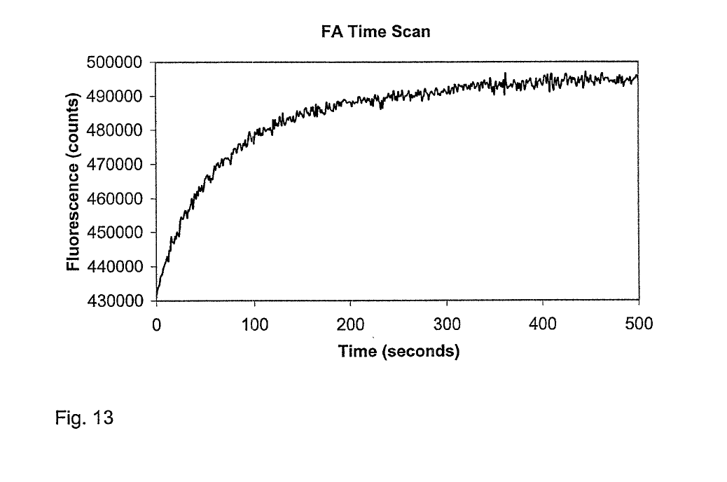

The fatty acyl-CoA synthetase activity was followed by measuring the increase in the fluorescence intensity al 460 nm in a cuvette containing the ACS reaction mixture, 3 μM FACI-24 and a GST-fusion of the E. coli ACS, FadD (FIG. 13). The results show that 460 nm emission increases in a linear fashion over time showing that FACI-24 is an excellent sensor for measuring ACS activity. The addition of the reaction mixture increases background fluorescence, however, this does not affect the sensitivity of the sensor.

Incubating GST-FadD in an ACS reaction mixture containing FACI-24 (3.0 μM) and increasing palmitic acid concentrations shows that FACI-24 produces an almost linear increase in 460 nm emission in response to the increased concentration of nonesterified fatty acid (NEFA) in the reaction mixture (FIG. 13). Thus, FACI-24 is an excellent sensor for establishing a very simple and highly sensitive assay for determining free fatty acid concentrations in biological fluids. The detection limit was fount to be <0.25 nmole/ml or 0.25 μM.

### EXAMPLE 9

### Determination of Free Fatty Acid Concentration in Biological Fluids

FA Assay Method:

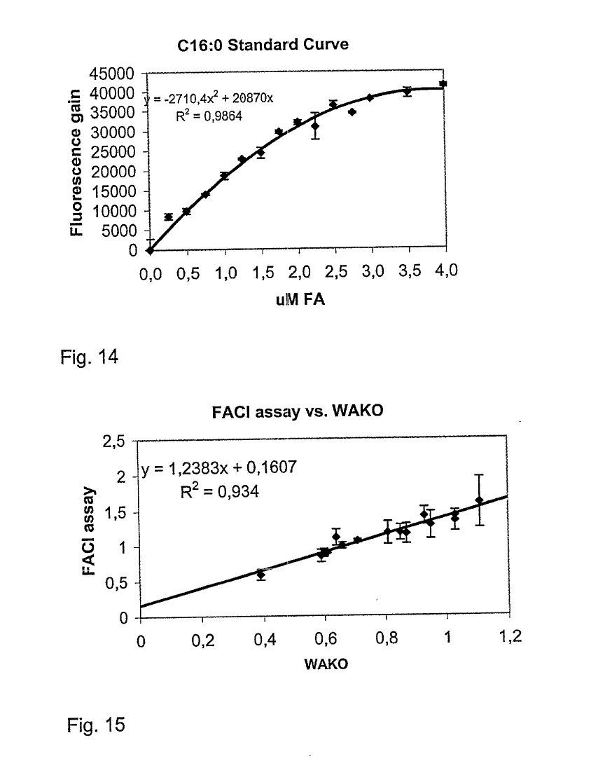

Fatty acids were extracted from 75 μl serum of blood by mixing with 925 μl of 96% ethanol, centrifuging for 2 minutes at 14,000 rpm and transferring ca. 850 μl to a new tube. Using 96-well black bottom-read microtiter plates, 5 μl extract was added to 200 μl reaction mixture (RM) (4 mM ATP, 4 mM MgCl₂, 500 μM EDTA, 3 μM bovine serum albumin, 3 μM FACI-24, 100 μM DTT, 60 μM CoASH, 200 mM Tris, pH 7.2). Three controls (C1-C3) were included on the plate; C1 was RM+ACS+5 μl ethanol, C2 was RM−ACS+5 μl ethanol and C3 was RM−ACS+5 μl extract. ACS activity was approximately 0.5 U per 200 μl. After 40 minutes in darkness and shaking the fluorescence upon excitation at 390 nm was measured at 460 nm using a Wallac 1420 multiwell reader. Fluorescence increase as result of fatty acids in the wells were calculated as follows: Fluorescence increase=(measurement−C1)−(C3−C2)=measurement−C1−C3+C2, taking into account the presence of ethanol, serum and ACS. The results are shown in FIG. 14.

This value obtained in this way compared to a standard curve prepared using a standard free fatty acid assay, NEFA C from WAKO Chemicals USA Inc. Richmond, Va., USA. The results of the comparison are shown in FIG. 15. The NEFA C kit is based on Acyl-CoA synthetase, Acyl-CoA oxidase coupled to a peroxidase assay.

### EXAMPLE 9

### Production of GST-FadD

Recombinant E. coli fatty acyl-CoA syntheatse was expressed as a N-terminal GST-fusion protein. The open reading frame of the E. coli fatty acyl-CoA syntheatse was amplified using the pN3576 plasmid as template (Black et al., 1997) and specific oligonucleotides 5′-CACGGATCCATGAAGMGGTTTGGCTTAACC-3′ and 5′-CACGAATTCTCAGGCTTTATTGTCCACTTTG-3′, carrying either a BamH1 and EcoR1 restriction site (underlined), respectively. The Expand High Fidelity PCR System was used as described by the manufacturer (Roche). The PCR product was digested with EcoR1 and BamH1 and ligated into the pGEX-2TK vector (Pharmacia) using standard techniques. The recombinant GST-fusion protein was expressed in E. coli BL21(DE3) strain and purified essentially as described by the manufacturer (Pharmacia), except that CoA (10 mM) was included in all buffers including the elution buffer.

1. A method for determination of the concentration of free unbound hydrophobic Coenzyme A ester in a sample comprising the steps of
i) providing a hydrophobic Coenzyme A binding construct exhibiting a first signal when unbound and exhibiting a measurably different second signal when bound to a hydrophobic Coenzyme A ester,
ii) contacting the sample with the labelled hydrophobic Coenzyme A binding construct,
iii) allowing at least one species of unbound free hydrophobic Coenzyme A ester to bind to the hydrophobic Coenzyme A binding construct forming a complex comprising a hydrophobic Coenzyme A ester and the hydrophobic Coenzyme A binding construct,
iv) detecting a signal from the complex,
v) correlating the signal to the concentration of the at least one species of hydrophobic Coenzyme A ester in the sample.

2. The method according to claim 1, wherein the hydrophobic Coenzyme A binding construct comprises a heterologous peptide capable of binding at least one species of hydrophobic Coenzyme A ester and a signal moiety.

3. The method according to claim 2, whereby heterologous peptide comprises an acyl-CoenzymeA binding protein, a variant or functional equivalent thereof.

4. The method according to claim 3, whereby the acyl-Coenzyme A binding protein comprises an amino acid sequence from the sequences of FIG. 1 (SEQ ID NO 1 to 30) a variant or functional equivalent thereof.

5. The method according to claim 2, whereby the heterologous peptide comprises bovine ACBP, a variant or functional equivalent thereof.

6. The method according to claim 2, whereby the heterologous peptide comprises a cystein or lysin residue for binding the signal moiety.

7. The method according to claim 6, whereby one native amino acid residue in the heterologous peptide has been substituted by a cystein or a lysin residue for binding the signal moiety.

8. The method according to claim 6, whereby the residue is selected from the amino acid residues aligning an acyl Coenzyme A binding domain.

9. The method according to claim 6, whereby the residue is selected from the amino acid residues having van der Waals' contact with a bound hydrophobic Coenzyme A ester.

10. The method according to claim 6, whereby the residue is selected from the amino acid residues being within 5 Å from a bound hydrophobic Coenzyme A ester.

11. The method according to claim 6, whereby the residue is selected from the amino acid residues making up the α-helices of the heterologous peptide.

12. The method according to claim 6, whereby the heterologous peptide comprises the bovine ACBP and the native amino acid being replaced by a cystein residue is selected from the group consisting of Met-24, Leu-25, Ala-53, Asp-21, Lys-50, Lys-54, Lys-18, pro-19, Ala-9, Tyr-31, Lys-32, Tyr-28, Tyr-73, Val-12, Lys-13, Leu-15; Ile-27; more preferably whereby the native amino acid is selected from the group consisting of Met-24, Ala-53, and Lys-50.

13. The method according to claim 1, whereby the complex formed during step iii) has a K_{D} below 2 μM, such as below 1.5 μM, for example below 1.0 μM, preferably below 500 nM, more preferably below 200 nM such as below 100 nM, for example below 90 nM, such as below 80 nM, for example below 70 nM, such as below 60 nM, for example below 50 nM, such as below 40 nM, for example below 30 nM, such as below 20 nM, for example below 15 nM, such as below 10 nM, for example below 8 nM, such as below 7 nM, for example below 6 nM, such as below 5 nM, for example below 4 nM, such as below 3 nM, for example below 2 nM, such as below 1 nM, for example below 0.5 nM, such as below 0.1 nM.

14. The method according to claim 13, whereby the complex formed during step iii) has a higher K_{D} with respect to other species of hydrophobic Coenzyme A esters.

15. The method according to claim 14, whereby the one species of hydrophobic Coenzyme A ester is selected from the group consisting of acyl Coenzyme A esters having a C2 acyl group, a C4 acyl group, a C6 acyl group, a C8 acyl group, a C10 acyl group, a C12 acyl group, a C14 acyl group, a C16 acyl group, a C18 acyl group, a C20 acyl group, a C22 acyl group, a C24 acyl group, a C26 acyl group, a saturated acyl group, a mono-unsaturated acyl group, a polyunsaturated acyl group, an acyl group comprising a cis double bond, an acyl group comprising a trans double bond, an acyl group comprising a ring structure, an acyl group comprising a side chain.

16. The method according to claim 1, whereby the signal comprises a fluorescence signal.

17. The method according to claim 1, whereby the detected signal is essentially proportional to the amount of hydrophobic Coenzyme A ester in the sample.

18. The method according to claim 17, whereby the detected signal is essentially proportional to the amount of at least one species of Coenzyme A ester in the sample.

19. The method according to claim 18, whereby the at least one species of Coenzyme A ester comprises a species selected from the group consisting of Coenzyme A esters with a C2 acyl group, a C4 acyl group, a C6 acyl group, a C8 acyl group a C10 acyl group, a C12 acyl group, a C14 acyl group, a C16 acyl group, a C18 acyl group, a C20 acyl group, a C22 acyl group, a C24 acyl group, a C26 acyl group, a saturated acyl group, a mono-unsaturated acyl group, a polyunsaturated acyl group, an acyl group comprising a cis double bond, an acyl group comprising a trans double bond, an acyl group comprising a ring structure, an acyl group comprising a side chain.

20. The method according to claim 1, whereby the detected signal from a first species of hydrophobic Coenzyme A ester is essentially 0 and the detected signal from a second species of hydrophobic Coenzyme A is essentially proportional to the amount of said second species in the sample.

21. The method according to claim 1, whereby the detected signal is essentially proportional to the amount of a group of hydrophobic Coenzyme A esters in the sample.

22. The method according to claim 1, further comprising a step prior to step ii) wherein hydrophobic acids in the sample are converted to hydrophobic Coenzyme A esters by acyl Coenzyme A ligase.

23. The method according to claim 21, further comprising a prior step wherein triacylglycerides in the sample are converted to glycerol and free fatty acids.

24. The method according to claim 21, further comprising a prior step wherein phospholipids in the sample are converted to glycerol and free fatty acids.

25. The method according to claim 1, whereby the sample is selected from the group consisting of blood, urine, milk, tears, faeces, sperm, cerebrospinal fluid, nasal secrete, food, feed and mixtures, dilutions, or extracts thereof.

26. A construct for binding hydrophobic Coenzyme A ester comprising
i) a heterologous peptide capable of binding at least one species of hydrophobic Coenzyme A ester,
ii) a signal moiety.

27. The construct according to claim 26, wherein the signal moiety comprises a fluorescent moiety.

28. The construct according to claim 26, wherein the signal moiety exhibits a first signal when the construct is unbound and a measurably different second signal when the construct is bound to a hydrophobic-Conenzyme A ester.

29. The construct according to 26, wherein the signal moiety comprises (6-bromoacetyl-2-dimethylaminonaphtalene) BADAN.

30. The construct according to claim 27, wherein the fluorescent moiety comprises a compound selected from the group consisting of acrylodan; 5-dimethylaminonaphtalene-1-sulfonyl aziridine (danzyl aziridine); 4-[N-[2-iodoacetoxy)ethyl]-N-methylamino]-7-nitrobenz-2-oxa 1,3 diazole ester (IANBDE); 4-[N-[2-iodoacetoxy)ethyl]-N-methylamino]-7-nitrobenz-2-oxa 1,3 diazole amide (IANBDA); 6-acryloyl-2-dimtheylaminonaphtalene (acrylodan); N-(7-chlorobenz-2-oxa-1,3-diazyl-4-yl)sulfonyl morpholine; 4-chloro-7-nitrobenz-2-oxa-1,3-diazole (NBD chloride); didansyl-L-cystine; N,N′-dimethyl-N-(iodoacetyl)-N′-(7-nitrobenz-2-oxa-1,3-diazol-4-yl)ethylenediamine (IANBD amide); 7-fluorobenz-2-oxa-1,3-diazole-4-sulfonamide (ABD-F); 4-fluoro-7-nitrobenz-2-oxa-1,3-diazole (NBD fluoride); 2-(4′-(iodoacetamido)anilino)naphtalene-6-sulfonic acid, sodium salt (IAANS); 5-(((2-iodoacetyl)amino)ethyl)amino)naphtalene-1-sulfonic acid (1,5-IAEDANS); 2-(4′-maleimidylanilino)naphtalene-6-sulfonic acid (MIANS); N-(1-pyreneethyl)iodoacetamide; N-(1-pyrene)iodoacetamide; N-(1-pyrene)maleimide; N-(1-pyrenemethyl)iodoacetamide (PMIA amide); 1-pyrenemethyl iodoacetate (PMIA ester); N-(1-pyrenepropyl)iodoacetamide); 1-(2,3-epoxypropyl)-4-(5-(4-methoxyphenyl)oxazol-2-yl)pyridinium trifluoromethanesulfonate (PyMPO epoxide); erythrosin-5-iodoacetamide; fluorescein-5-maleimide; 5-iodoacetamidofluorescein (5-IAF); 6-iodoacetamidofluorescein (6-IAF); 1-(2-maleimidylethyl)-4-(5-(4-methoxyphenyl)oxazol-2-yl)pyridinium methanesulfonate (PyMPO maleimide); Oregon Green™ 488 iodoacetamide “mixed isomers”; tetramethylrhodamine-5-iodoacetamide (5-TMRIA) “single isomer”; tetramethylrhodamine-5-maleimide “single isomer”; tetramethylrhodamine-6-maleimide “single isomer”; Texas Red® C₅ bromoacetamide; Texas Red® C₂ maleimide;

31. The construct according to claim 26, further comprising a second signal moiety.

32. The construct according to claim 31, wherein the second signal moiety is selected from the group of claim 30.

33. The construct according to claim 26, wherein the heterologous peptide comprises an acyl-CoenzymeA binding protein, a variant or functional equivalent thereof.

34. The construct according to claim 33, wherein the acyl-Coenzyme A binding protein comprises an amino acid sequence from the sequences of FIG. 1 (SEQ ID NO 1 to 30) a variant or functional equivalent thereof.

35. The construct according to claim 26, wherein the heterologous peptide comprises an acyl-CoenzymeA binding domain.

36. The construct according to claim 26, wherein the heterologous peptide comprises bovine ACBP, a variant or functional equivalent thereof.

37. The construct according to claim 26, wherein the signal moiety is bound to a cystein or a lysin residue comprised in the heterologous peptide.

38. The construct according to claim 37, wherein the residue is non-native to the peptide.

39. The construct according to claim 37, wherein the residue is selected from the amino acid residues aligning an acyl Coenzyme A binding domain.

40. The construct according to claim 37, wherein the residue is selected from the amino acid residues being within 5 Å from a bound hydrophobic Coenzyme A ester.

41. The construct according to claim 37, wherein the residue is selected from the amino acid residues making up α-helices of the heterologous peptide.

42. The construct according to the claim 38, wherein the heterologous peptide comprises the bovine ACBP and the native amino acid being replaced by a cystein residue is selected from the group consisting of Met-24, Leu-25, Ala-53, Asp-21, Lys-50, Lys-54, Lys-18, pro-19, Ala-9, Tyr-31, Lys-32, Tyr-28, Tyr-73, Val-12, Lys-13, Leu-15; Ile-27; more preferably wherein the native amino acid is selected from the group consisting of Met-24, Ala-53, and Lys-50.

43. The construct according to claim 26, having a K_{D} with respect to at least one hydrophobic Coenzyme A ester below 2 μM, such as below 1.5 μM, for example below 1.0 μM, preferably below 500 nM, more preferably below 200 nM such as below 100 nM, for example below 90 nM, such as below 80 nM, for example below 70 nM, such as below 60 nM, for example below 50 nM, such as below 40 nM, for example below 30 nM, such as below 20 nM, for example below 15 nM, such as below 10 nM, for example below 8 nM, such as below 7 nM, for example below 6 nM, such as below 5 nM, for example below 4 nM, such as below 3 nM, for example below 2 nM, such as below 1 nM, for example below 0.5 nM, such as below 0.1 nM.

44. The construct according to claim 43, having a K_{D} with respect to one species of hydrophobic Coenzyme A ester below 2 μM, such as below 1.5 μM, for example below 1.0 μM, preferably below 500 nM, more preferably below 200 nM, such as below 100 nM, for example below 90 nM, such as below 80 nM, for example below 70 nM, such as below 60 nM, for example below 50 nM, such as below 40 nM, for example below 30 nM, such as below 20 nM, for example below 15 nM, such as below 10 nM, for example below 8 nM, such as below 7 nM, for example below 6 nM, such as below 5 nM, for example below 4 nM, such as below 3 nM, for example below 2 nM, such as below 1 nM, for example below 0.5 nM, such as below 0.1 nM and a higher K_{D} with respect to other species of hydrophobic Coenzyme A esters.

45. The construct according to claim 44, wherein the one species of hydrophobic Coenzyme A ester is selected from the group consisting of acyl Coenzyme A esters having a C2 acyl group, a C4 acyl group, a C6 acyl group, a C8 acyl group, a C10 acyl group, a C12 acyl group, a C14 acyl group, a C16 acyl group, a C18 acyl group, a C20 acyl group, a C22 acyl group, a C24 acyl group, a C26 acyl group, a saturated acyl group, a mono-unsaturated acyl group, a polyunsaturated acyl group, an acyl group comprising a cis double bond, an acyl group comprising a trans double bond, an acyl group comprising a ring structure, an acyl group comprising a side chain.

46. A kit for detection of the concentration of a hydrophobic Coenzyme A ester in a sample comprising
i) at least a first construct according to claims 26 to 45,
ii) a sample compartment for application of the sample.

47. The kit according to claim 46, further comprising an acyl-Coenzyme A synthetase, coenzyme A, adenosinetriphosphate, Mg⁺⁺, an antioxidant, and buffer.

48. The kit according to claim 47, further comprising pyrophosphatase.

49. The kit according to claim 47, further comprising a lipase, and buffer.

50. The kit according to claim 47, further comprising a phospholipase such as phospholipase A1 and/or A2, and buffer.

51. The kit according to claim 46, further comprising albumin.

52. The kit according to claim 46, wherein compounds are freeze dried.

53. The kit according to claim 46, wherein the hydrophobic-Coenzyme A ester binding construct is immobilised.

54. The kit according to claim 53, wherein the construct is immobilised in at least two different places, such as at least 3, for example at least 4 such as at least 5 different spaces.

55. The kit according to claim 46, comprising a second hydrophobic-Coenzyme A ester binding construct according to claims 26 to 45.

56. The kit according to claim 55, further comprising at least a third construct, such as at least a third and a fourth construct, for example at least a third, a fourth and a fifth construct.

57. The kit according to claim 55 or 56, wherein each construct has a K_{D} with respect to at least one species or a group of species of hydrophobic Coenzyme A esters, which is substantially lower than the K_{D} of the other construct(s) with respect to this species or group of species.

58. The kit according to claim 57, wherein substantially lower is 10 times lower, preferably 100 times lower.

59. The kit according to claim 55, wherein the first construct is a fluorescence acyl-CoA sensor 1 (FACI 24) and the second construct is a fluorescence acyl-CoA sensor 2 (FACI 53).

60. A method for determining the amount of free hydrophobic carboxylic acid(s) and/or lipid constituents in a sample comprising
i. optionally fractionating the sample to obtain a substantially cell-free sample,
ii. mixing the substantially cell-free sample with an amount of water-miscible organic solvent to precipitate proteins and obtain a solution of free fatty acids,
iii. subjecting a sample of the supernatant to a quantitative analysis determining the amount of free fatty acids in the sample.

61. The method according to claim 60, wherein the sample comprises a blood sample and the substantially cell-free sample is serum.

62. The method according to claim 60, wherein the alcohol comprises a low molecular weight alcohol selected from the group consisting of ethanol, methanol, 1-propanol, 2-propanol, cyclopropanol.

63. The method according to claim 60, whereby the low molecular weight alcohol is selected from the group consisting of ethanol and 1-propanol.

64. The method according to claim 60, whereby the low molecular weight alcohol is ethanol.

65. The method according to claim 60, wherein step iii) comprises diluting a sub-sample of the solvent comprising the free fatty acids in a reaction mixture and performing a method according to any of claims 1 to 25.

66. The method according to claim 60, wherein step iii) comprises gas-chromatography, HPLC, or binding to a fluorescently modified fatty acid binding protein.


SEQUENCE LISTING (38 sequences)

SEQ ID NO: 1 - protein, 86 aa, Bos taurus
SQAEFDKAAEEVKHLKTKPADEEMLFIYSHYKQATVGDINTERPGMLDFKGKAKWDAWNELKGTSKEDAMKAYIDKVEELKKKYGI

SEQ ID NO: 2 - protein, 86 aa, Homo sapiens [sequence omitted]

SEQ ID NO: 3 - protein, 86 aa, Sus scrofa
SQAEFEKAAEEVKNLKTKPADDEMLFIYSHYKQATVGDINTERPGILDLKGKAKWDAWNGLKGTSKEDAMKAYINKVEELKKKYGI

SEQ ID NO: 4 - protein, 86 aa, Canis familiaris
SQAEFDKAAEDVKHLKTKPADDEMLYIYSHYKQATVGDINTERPGLLDLRGKAKWDAWNQLKGTSKEDAMKAYVNKVEDLKKKYGI

SEQ ID NO: 5 - protein, 86 aa, Rattus norvegicus
SQADFDKAAEEVKRLKTQPTDEEMLFIYSHFKQATVGDVNTDRPGLLDLKGKAKWDSWNKLKGTSKENAMKTYVEKVEELKKKYGI

SEQ ID NO: 6 - protein, 86 aa, Mus musculus
SQAEFDKAAEEVKRLKTQPTDEEMLFIYSHFKQATVGDVNTDRPGLLDLKGKAKWDSWNKLKGTSKESAMKTYVEKVDELKKKYGI

SEQ ID NO: 7 - protein, 86 aa, Terrapene carolina
SQAEFDKAAEEVKQLKSQPTDEEMLYIYSHFKQATVGDINTERPGFLDFKGKAKWDAWDALKGMAKEEAMKAYIAKVEELKGKYGI

SEQ ID NO: 8 - protein, 86 aa, Anas platyrhynchos
AEAAFQKAAEEVKQLKSQPSDQEMLDVYSHYKQATVGDVNTDRPGMLDFKGKAKWDAWNALKGMSKEDAMKAYVAKVEELKGKYGI

SEQ ID NO: 9 - protein, 86 aa, Gallus gallus
SEAAFQKAAEEVKELKSQPTDQEMLDVYSHYKQATVGDVNTDRPGMLDFKGKAKWDAWNALKGMSKEDAMKAYVAKVEELKGKYGI

SEQ ID NO: 10 - protein, 85 aa, Drosophila melanogaster
VSEQFNAAAEKVKSLTKRPSDDEFLQLYALFKQASVGDNDTAKPGLLDLKGKAKWEAWNKQKGKSSEAAQQEYITFVEGLVAKYA

SEQ ID NO: 11 - protein, 88 aa, Manduca sexta
LQEQFDQAASNVRNLKSLPSDNDLLELYALFKQASAGDADPANRPGLLDLKGKAKFDAWHKKAGLSKEDAQKAYIAKVESLIASLGLQ

SEQ ID NO: 12 - protein, 86 aa, Saccharomyces cerevisiae
VSQLFEEKAKAVNELPTKPSTDELLELYALYKQATVGDNDKEKPGIFNMKDRYKWEAWENLKGKSQEDAEKEYIALVDQLIAKYSS

SEQ ID NO: 13 - protein, 86 aa, Saccharomyces monoasensis
VSQLFEEKAKAVNELPTKPSTDELLELYGLYKQATVGDNDKEKPGIFNMKDRYKWEAWEDLKGKSQEDAEKEYIAYVDNLIAKYSS

SEQ ID NO: 14 - protein, 86 aa, Caenorhabditis elegans
MTLSFDDAAATVKTLKTSPSNDELLKLYALFKQGTVGDNTTDKPGMFDLKGKAKWSAWDEKKGLAKDDAQKAYVALVEELIAKYGA

SEQ ID NO: 15 - protein, 86 aa, Gossypium hirsutum
LKEEFEEHAEKVKTLPAAPSNDDMLILYGLYKQATVGPVNTSRPGMFNMREKYKWDAWKAVEGKSKEEAMGDYITKVKQLFEAAGS

SEQ ID NO: 16 - protein, 86 aa, Brassica napus
LKEDFEEHAEKVKKLTASPSNEDLLILYGLYKQATVGPVTTSRPGMFSMKERAKWDAWKAVEGKSTDEAMSDYITKVKQLLEAEAS

SEQ ID NO: 17 - protein, 86 aa, Arabidopsis thaliana
LKEEFEEHAEKVNTLTELPSNEDLLILYGLYKQAKFGPVDTSRPGMFSMKERAKWDAWKAVEGKSSEEAMNDYITKVKQLLEVEAS

SEQ ID NO: 18 - protein, 86 aa, Ricinus communis
LKEDFEEHAEKAKTLPENTTNENKLILYGLYKQATVGPVNTSRPGMFNMRDRAKWDAWKAVEGKSTEEAMSDYITKVKQLLGEAAA

SEQ ID NO: 19 - protein, 85 aa, Lilium sp.
LKEEFEEHAVKAKTLPESTSNENKLILYGLYKQSTVGPVDTGRPGMFSPRERAKWDAWKAVEGKSKEEAMGDYITKVKQLLEESA

SEQ ID NO: 20 - protein, 86 aa, Rana sp.
PQADFDKAAGDVKKLKTKPTDDELKELYGLYKQSTVGDINIECPGMLDLKGKAKWDAWNLKKGLSKEDAMSAYVSKAHELIEKYGL

SEQ ID NO: 21 - protein, 86 aa, Anas platyrhynchos
HQADFDEAAEEVKKLKTRPTDEELKELYGFYKQATVGDINIECPGMLDLKGKAKWEAWNLKKGISKEDAMNAYISKAKTMVEKYGI

SEQ ID NO: 22 - protein, 86 aa, Bos taurus
CQVEFEMACAAIKQLKGPVSDQEKLLVYSYYKQATQGDCNIPAPPATDLKAKAKWEAWNVEKGMSKMDAMRIYIAKVEELKKNEAG

SEQ ID NO: 23 - protein, 86 aa, Rattus norvegicus
SQVEFEMACASLKQLKGPLSDQEKMLVYSFYKQATQGDCNIPVPPATDVKAKAKWEAWMVNKGMSKMDAMRIYIAKVEELKKNETC

SEQ ID NO: 24 - protein, 86 aa, Mus musculus
SQVEFEMACASLKQLKGPVSDQEKLLVYSFYKQATQGDCNIPVPPATDVRAKAKYEAWMVNKGMSKMDAMRIYIAKVEELKKKEPC

SEQ ID NO: 25 - protein, 85 aa, Caenorhabditis elegans
AQADFEKAQKNLKTLKEEPDNDVKLQLYGLFKQATAGDVQGKRPGMMDFVGRAKYDAWNTLKGQTQDEARANYAKLVGGLISEEA

SEQ ID NO: 26 - protein, 90 aa, Caenorhabditis elegans
LQEKFDAAVEIIQKLPKTGPVATSNDQKLTFYSLFKQASIGDVNTDRPGIFSIIERKKWDSWKELEGVSQDEAKERYIKALNDMFDKIAE

SEQ ID NO: 27 - protein, 90 aa, Caenorhabditis elegans
LDEQFEAAVWIINALPKNGPIKTSINDQLQMYSLYKQATSGKCDTIQPYFFQIEQRMKWNAWNQLGNMDEAEAKAQYVEKMLKLCNQAEA

SEQ ID NO: 28 - protein, 85 aa, Cyprinus carpio
SVEEFNAAKEKLGALKKDPGNEVKLKVYALFKQATQGPCNTPKPSMLDFVNKAKWDAWKSLGSVSQEEARQQYVDLISSLVGTEA

SEQ ID NO: 29 - protein, 89 aa, Bos taurus
HETRFEAAVKVIQSLPKNGSFQPTNEMMLKFYSFYKQATEGPCKLSKPGFWDPVGRYKWDAWSSLGDMTKEEAMIAYVEEMKKILETMP

SEQ ID NO: 30 - protein, 86 aa, Arabidopsis thaliana
SAATAFVAAAASDRLSQKVSNELQLQLYGLYKIATEGPCTAPQPSALKMTARAKWQAWQKLGAMPPEEAMEKYIDLVTQLYPAWVE

SEQ ID NO: 31 - DNA, 27 bp, Artificial Sequence
  M24C primer upstream
tgcttgttcatctactctcactacaag

SEQ ID NO: 32 - DNA, 25 bp, Artificial Sequence
  M24C primer downstream
ttcttcgtcggccggcttggtcttc

SEQ ID NO: 33 - DNA, 27 bp, Artificial Sequence
  M46C primer upstream
tgcttggacttcaagggtaaggctaag

SEQ ID NO: 34 - DNA, 24 bp, Artificial Sequence
  M46C primer downstream
cccgggtctttcggtgttgatgtc

SEQ ID NO: 35 - DNA, 27 bp, Artificial Sequence
  A53C primer upstream
tgcaagtgggacgcttggaacgaattg

SEQ ID NO: 36 - DNA, 24 bp, Artificial Sequence
  A53C primer downstream
cttacccttgaagtccaacatccc

SEQ ID NO: 37 - DNA, 31 bp, Artificial Sequence
  synthetic oligonucleotide for amplifying E. coli fatty acyl-CoA synthetase ORF, with BamHI site
cacggatccatgaagaaggtttggcttaacc

SEQ ID NO: 38 - DNA, 31 bp, Artificial Sequence
  synthetic oligonucleotide for amplifying E. coli fatty acyl-CoA synthetase ORF, with EcoRI site
cacgaattctcaggctttattgtccactttg